(19) United States
(12) Patent Application Publication
(10) Pub. No.: US 2026/0363754 A1
(43) Pub. Date: Nov. 19, 2026

(54) SYSTEM, DEVICES, AND METHODS FOR DELIVERING RADIOFREQUENCY CURRENT FOR TISSUE PERFORATION

(71) Applicant: ATRAVERSE MEDICAL, INC., Cardiff, CA (US)

(72) Inventors: Jay Lee KELLEY, Cardiff, CA (US); Steven Richard MICKELSEN, Cardiff, CA (US); Eric Douglas SAUTER, Cardiff, CA (US); Michael C. GARRETT, Northbrook, IL (US)

(21) Appl. No.: 19/754,407

(22) Filed: Jul. 27, 2026

Related U.S. Application Data
(63) Continuation of application No. PCT/US25/13215, filed on Jan. 27, 2025.
(60) Provisional application No. 63/625,845, filed on Jan. 26, 2024.
(60) Provisional application No. 63/667,367, filed on Jul. 3, 2024.

(51) Int. Cl. A61B 18/12; A61B 18/00; A61B 18/14

(52) U.S. Cl. CPC A61B 18/1206; A61B 18/1492; A61B 2018/00083; A61B 2018/00357; A61B 2018/00577; A61B 2018/00767; A61B 2018/00827; A61B 2018/00875; A61B 2018/00892

(57) ABSTRACT

An apparatus including an electrosurgical device, including a distal conductive region configured to puncture through a tissue wall, and a generator configured to couple to the electrosurgical device, including a memory and a processor operatively coupled to the memory. The processor configured to deliver a voltage waveform to the distal conductive region of the electrosurgical device, while delivering the voltage waveform, monitor one or more electrical parameters via the distal conductive region, determine a plurality of states of the distal conductive region over a period of time based on the one or more electrical parameters, determine whether at least one state of the plurality of states satisfies a condition indicative of the one or more electrical parameters being outside of a predefined range, and in response to determining that the at least one state of the plurality of states satisfies the condition, terminate the delivery of the voltage waveform.

### CROSS-REFERENCE TO RELATED APPLICATIONS

This application is a continuation of International Patent App. No. PCT/US2025/013215, entitled “SYSTEM, DEVICES, AND METHODS FOR DELIVERING RADIOFREQUENCY CURRENT FOR TISSUE PERFORATION,” filed Jan. 27, 2025, the disclosure of which is incorporated by reference herein, in its entirety. International Patent App. No. PCT/US2025/013215 claims priority to and benefit of U.S. Provisional Patent Application No. 63/625,845, titled “SYSTEMS, DEVICES, AND METHODS FOR DELIVERING RADIOFREQUENCY CURRENT FOR TISSUE PERFORATION,” filed Jan. 26, 2024, and U.S. Provisional Patent Application No. 63/667,367, titled “SYSTEMS, DEVICES, AND METHODS FOR DELIVERING RADIOFREQUENCY CURRENT FOR TISSUE PERFORATION,” filed Jul. 3, 2024, the disclosures of which are incorporated by reference herein, in their entirety.

### TECHNICAL FIELD

The embodiments described herein relate generally to medical devices for delivering radiofrequency (RF) current to a guidewire for crossing an inter-atrial septum.

### BACKGROUND

In many medical procedures, it may be necessary to puncture through a tissue structure to gain access to treatment sites or create passageways or connections between different anatomical structures. For example, in cardiac interventions, a needle, catheter, or guidewire is commonly used to puncture through the atrial septum to gain access to the left side of the heart, e.g., for evaluating or treating cardiac anomalies. In some instances, the guidewire or catheter can be equipped with an energy delivery device that can deliver energy such as radiofrequency (RF) energy to a tissue structure, such as the septum, to perforate through it.

While there are existing systems capable of perforating and crossing through tissue structures, such systems suffer from various drawbacks. For example, such systems may cause unwanted injury to other areas of the heart from inadvertent perforation or lead to char or thrombus formation due to high operating temperatures. Therefore, there exists further improvements to such systems.

### SUMMARY

In some embodiments, an apparatus includes: an electrosurgical device including a distal conductive region configured to puncture through a tissue wall, and a generator configured to couple to the electrosurgical device. The generator includes: a memory and a processor operatively coupled to the memory. The processor configured to deliver a voltage waveform to the distal conductive region of the electrosurgical device, while delivering the voltage waveform, monitor one or more electrical parameters via the distal conductive region, determine a plurality of states of the distal conductive region over a period of time based on the one or more electrical parameters, determine whether at least one state of the plurality of states satisfies a condition indicative of the one or more electrical parameters being outside of a predefined range, and in response to determining that the at least one state of the plurality of states satisfies the condition, terminate the delivery of the voltage waveform to the distal conductive region.

In some embodiments, an apparatus includes: an electrosurgical device including a distal conductive region configured to puncture through a tissue wall. The distal conductive region has a length of at least about 1 cm. The apparatus includes: a generator configured to couple to the electrosurgical device. The generator includes: a memory and a processor operatively coupled to the memory. The processor is configured to: deliver a voltage waveform to the distal conductive region of the electrosurgical device, while delivering the voltage waveform, monitor an electrical parameter of an circuit including the distal conductive region, determine, based on the electrical parameter, whether a condition indicative of the electrical parameter being outside of a predefined range, and in response to determining that the condition is satisfied, terminate the delivery of the voltage waveform to the distal conductive region.

In some embodiments, a system includes an electrosurgical device including a distal conductive region configured to deliver energy to cross through a tissue wall. The distal conductive region has a length of at least about 1 cm. The system includes a generator configured to couple to the electrosurgical device. The generator includes: a memory and a processor operatively coupled to the memory. The processor configured to: deliver a voltage waveform to the distal conductive region to cause the delivery of energy, while delivering the voltage waveform, measure voltage and current signals over time via the distal conductive region, determine, based on the voltage and current signals, an impedance over time associated with the distal conductive region, determine, based on the impedance over time and during a delivery of energy to the distal conductive region, a state of the distal conductive region, the state corresponding to a position of the distal conductive region relative to a surface of the tissue wall, and terminate, when the state of the distal conductive region is indicative of a failure mode or a successful crossing of the tissue wall, the delivery of the voltage waveform to the distal conductive region.

In some embodiments, a method includes: delivering a voltage waveform to a distal conductive region of an electrosurgical device, the distal conductive region having a length of at least about 1 cm, measuring, while delivering the voltage waveform and while the distal conductive region is advanced toward a tissue wall, one or more electrical parameters via the distal conductive region, determining, based on the one or more electrical parameters, one or more states of the distal conductive region over time, the one or more states corresponding to a position of the distal conductive region relative to a surface of the tissue wall, determining that the one or more states or a sequence of the one of more states satisfies a condition that is indicative of a failure mode or a successful crossing of the tissue wall, and in response to determining that the condition is satisfied, terminating the delivery of the voltage waveform to the distal conductive region.

In some embodiments, a method includes: delivering a voltage waveform to a distal conductive region of an electrosurgical device, the distal conductive region having a length of at least about 1 cm, monitoring, while delivering the voltage waveform, an electrical parameter of a circuit including the distal conductive region, determining, based on the electrical parameter, whether a condition indicative of the electrical parameters being outside of a predefined range, and in response to determining that the condition is satisfied, terminate the delivery of the voltage waveform to the distal conductive region.

### BRIEF DESCRIPTION OF FIGURES

FIG. 1A is a schematic diagram of an electrosurgical system, according to embodiments.

FIG. 1B schematically depicts an example generator for delivering radiofrequency (RF) energy to an electrosurgical device, according to embodiments.

FIG. 2 is a schematic diagram of an electrosurgical system, showing components positioned relative to a patient, according to embodiments.

FIG. 3A depicts an example of a guidewire of an electrosurgical device, according to embodiments. FIGS. 3B and 3C depict the operation of the guidewire of FIG. 3A in crossing a tissue structure, according to embodiments.

FIGS. 4A-4C depict perspective views of a guidewire of an electrosurgical device, according to embodiments.

FIGS. 5A-5B depict an unintentional lesion being formed by an electrosurgical device in a heart of a patient, according to embodiments.

FIG. 6 is a flow chart depicting a method of using the electrosurgical system, according to embodiments.

FIG. 7 is a flow chart depicting a method of varying a RF output being delivered to a guidewire, according to embodiments.

FIG. 8 is a flow chart depicting a method of delivering a RF output to a guidewire, according to embodiments.

FIG. 9 is as flow chart depicting a method of adjusting energy delivered to an electrosurgical device, according to embodiments.

FIG. 10 depicts different states of a distal end of an electrosurgical device used for perforating tissue, according to embodiments.

FIG. 11 depicts various embodiments of state transitions, according to embodiments.

FIG. 12 depicts a graph of resistance as measured by a generator as an electrosurgical device engages tissue, according to an embodiment.

FIG. 13 depicts a flow diagram of a method for operating a generator, according to an embodiment.

FIGS. 14A-14B depict representative circuits of an electrosurgical device and tissue, according to embodiments.

FIGS. 15A-15M depict detected and determined values associated with an electrosurgical device engaging tissue, according to embodiments.

FIGS. 16A-16I depict detected and determined values associated with an electrosurgical device engaging tissue, according to embodiments.

FIG. 17 depicts an example guidewire of an electrosurgical device, according to embodiments.

FIG. 18A depicts required RMS voltage to deliver a real power, with a fixed upper limit, and FIG. 18B depicts real power as a function of Rp, with a fixed voltage upper limit.

### DETAILED DESCRIPTION

Described in various embodiments herein are systems, devices, device components, and methods for delivering radiofrequency (RF) energy to a guidewire (e.g., single-use, disposable guidewire) for crossing thin tissue structures for biomedical applications such as atrial crossing. In particular, electrosurgical systems are configured to puncture and cross thin tissue structures for biomedical applications. Aspects of these embodiments may provide for a safer, faster or more convenient tissue puncture.

An example use for electrosurgical systems, assemblies and devices as described herein is to facilitate transseptal puncture procedures (e.g., atrial crossing). Performing transseptal puncture, also known as atrial crossing, is a necessary procedural step for a myriad of cardiac interventions, including cardiac ablation for treatment of arrythmias such as atrial fibrillation and atrial flutter, occlusion of the left atrial appendage, and transcatheter repair of the mitral valve. These diseases and others affect millions of people worldwide. For transcatheter therapies of the left side of the heart, a direct pathway from the large diameter vena cava can be utilized to deliver large bore sheaths and devices (e.g., about 8-20 French) to the right atrial chamber. After the catheter is delivered to the right side of the heart, a small puncture is then made in the interatrial septum dividing the left and right sides of the heart to gain access to the left side of the heart. The inter atrial septum is composed of a thin fibrous structure known as the fossa ovalis (FO).

For example, an electrosurgical system described herein can include a device, such as a generator, that delivers RF current to a guidewire for crossing the inter-atrial septum. The electrosurgical system can be configured to monitor the state of the guidewire before, during, and after the crossing of the septum based on characteristics of the energy delivered by the generator. This monitoring allows the RF generator to react to the various states and adjust the power output, including discontinuing the RF, based on the various possible states. Adjusting the power output decreases the likelihood of the guidewire causing undesired damage to the heart.

Further details of the electrosurgical systems, devices, and methods described herein are provided in the sections below.

### Overview of Electrosurgical Systems and Devices

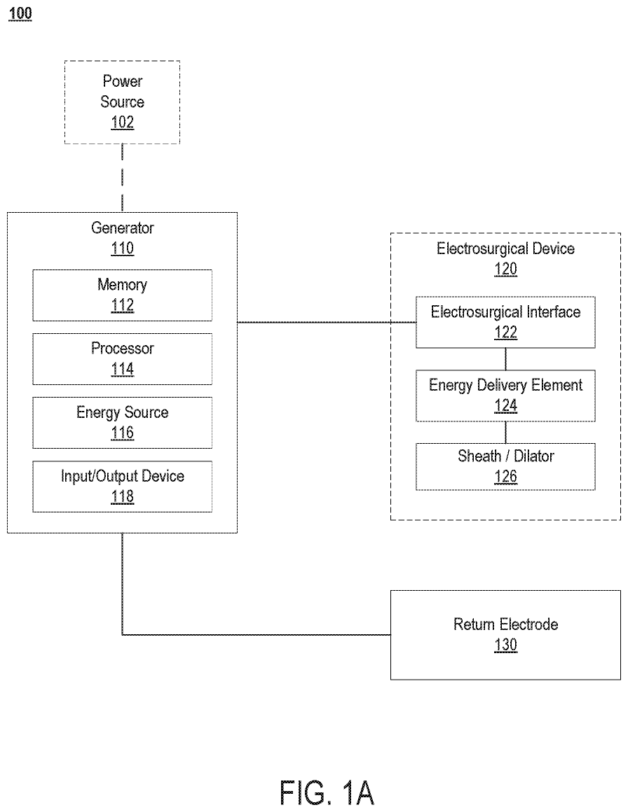

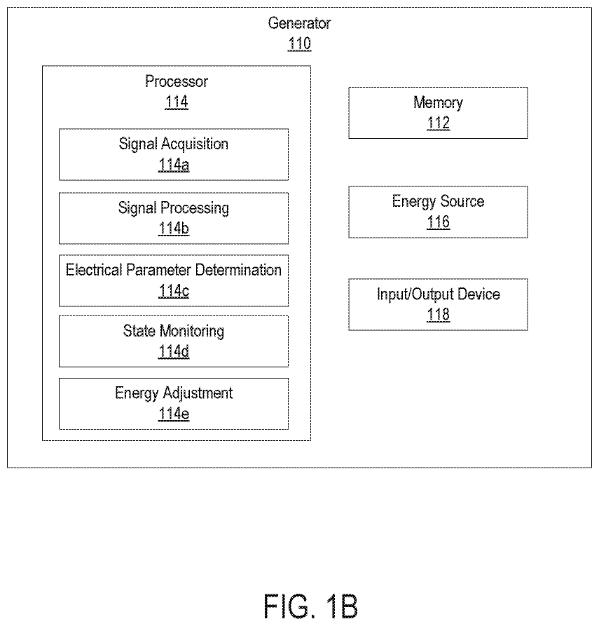

FIG. 1A is a schematic diagram of an electrosurgical system 100, according to embodiments. The electrosurgical system 100 includes a generator 110, an electrosurgical device or assembly 120, and a return electrode 130. Electrosurgical device or assembly 120 can include an electrosurgical interface 122, an energy delivery element 124, and a sheath or dilator 126.

The generator 110 can be configured to generate energy, such as, for example, radiofrequency (RF) energy. The generator 110 can include a memory 112, a processor 114, an energy source 116, and an input/output device 118. In some embodiments, the generator 110 can be coupled to an external power source 102, such as, for example, a direct current (DC) power supply. The generator 110 can include a plug or adaptor, which can be used to plug into a socket. Alternatively, or additionally, the generator 110 can include an onboard power source, such as, for example, a battery.

The memory 112 may include a database (not shown) and may be, for example, a random access memory (RAM), a memory buffer, a hard drive, an erasable programmable read-only memory (EPROM), an electrically erasable read-only memory (EEPROM), a read-only memory (ROM), Flash memory, etc. The memory 112 may store instructions to cause the processor 114 to execute modules, processes and/or functions associated with the system 100, such as voltage waveform generation and/or impedance monitoring, as further described below.

The processor 114 can be any suitable processing device configured to run and/or execute a set of instructions or code. The processor may be, for example, a general purpose processor, a Field Programmable Gate Array (FPGA), an Application Specific Integrated Circuit (ASIC), a Digital Signal Processor (DSP), and/or the like. The processor may be configured to run and/or execute application processes and/or other modules, processes and/or functions associated with the system and/or a network associated therewith (not shown). The underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like. The generator 110 is further described in reference to FIG. 1B.

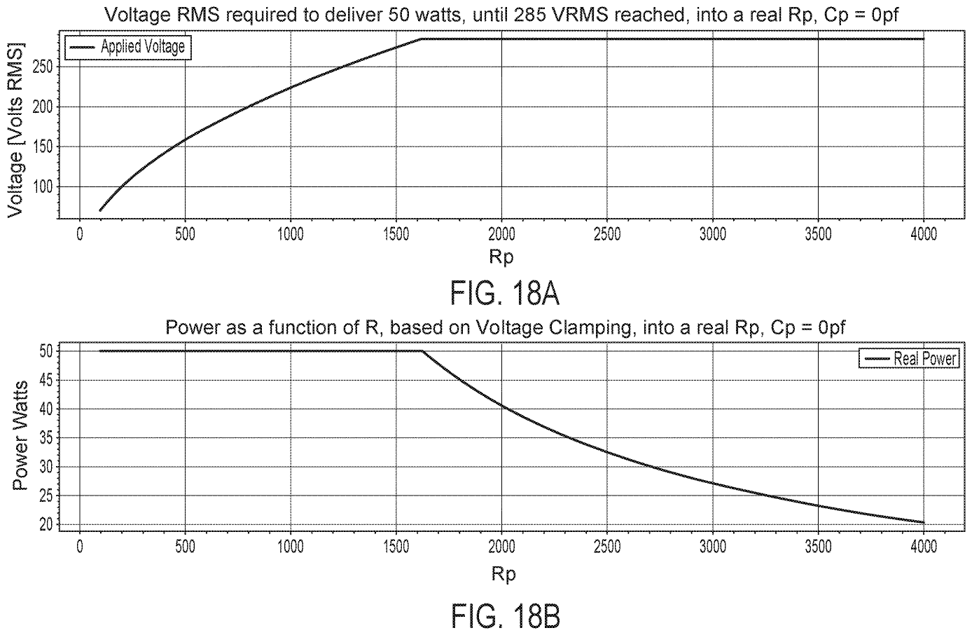

The energy source 116 can be configured to convert, store, and/or supply energy, e.g., in the form of a voltage waveform. In some embodiments, the energy source 116 can include an alternating current (AC)/DC switcher. In some embodiments, the energy source 116 can include one or more capacitors to store energy from a power supply. In some embodiments, the energy source 116 can be configured to generate and deliver voltage waveforms, e.g., to the electrosurgical device 120. In some embodiments, the voltage waveform can be an oscillating sinusoidal RF waveform. The voltage waveform can have a frequency of between about 200 kHz to about 1 MHz, including all sub-ranges and values therebetween. For example, the voltage waveform can have a frequency of between about 350 kHz and about 500 kHz, or a frequency of about 470 kHz, in some applications. The voltage waveform can have a peak voltage of between about 25 V and about 400 V, root mean square (RMS), including all sub-ranges and values therebetween. For example, the voltage waveform can have a peak voltage of between about 150 V to about 300 V, or a peak voltage of about 285 V, in some applications. As seen in FIG. 18A-18B, the generator 110 can be configured to output power until a fixed (e.g., predetermined) voltage is reached.

The input/output device 118 can be configured to provide a communication interface between an operator and the system 100. The input/output device 118 can include one or more input devices and output devices. In some embodiments, an input device of the input/output device 118 may include a touchscreen or other touch-sensitive device, a step switch, a foot pedal, a keypad, a keyboard, a button, a joystick, etc. In some embodiments, an output device of the input/output device 118 may include one or more of a display device and audio device. The display device may include at least one of a light emitting diode (LED), liquid crystal display (LCD), electroluminescent display (ELD), plasma display panel (PDP), thin film transistor (TFT), and organic light emitting diodes (OLED). An audio device may audibly output patient data, sensor data, system data, other data, alarms, warnings, and/or the like. The audio device may include at least one of a speaker, piezoelectric audio device, magnetostrictive speaker, and/or digital speaker.

The generator 110 can be coupled to the electrosurgical device 120 and the return electrode 130. In use, the signal generator 110 is configured to generate voltage waveforms for puncturing through tissue, such as, for example, the atrial septum. For example, the generator 110 may be configured to generate and deliver a voltage waveform to the electrosurgical device 120. The return electrode 130 may be coupled to a patient (e.g., disposed on a patient's back, torso, or extremity such as a leg) to allow current to pass from the electrosurgical device 120 through the patient and then to the return electrode 130 to provide a safe current return path from the patient.

In some embodiments, the generator 110 can operate in a constant power mode with a typical setting of about 5 to about 50 W. In some embodiments, the generator 110 can implement a feedback loop control, e.g., via a controller of the generator or a controller operatively coupled to the generator. In some embodiments, the generator 110 can be configured to monitor the voltage and/or current outputted by the generator 110. In some embodiments, the generator 110 (or controller) can include circuitry configured to determine an impedance and/or other parameters of the circuit going from the generator 110 to the electrosurgical device 120 and to the return electrode 130 and back to the generator 110.

In some embodiments, the generator 110 can be configured to implement an automatic energy shut off algorithm. For example, the generator 110, while delivering energy to the electrosurgical device 120, can be configured to monitor one or more parameters associated with a distal end of the electrosurgical device 120 and, based on monitoring the parameters, determine to terminate the delivery of energy. Alternatively, or additionally, the generator 110 can be configured to modulate the delivery of energy based on monitoring one or more parameters associated with the distal end of electrosurgical device 120. In some embodiments, the processor 114 or other processing circuitry within the generator 110 can be configured to monitor parameters, such as impedance, power, etc., associated with a procedure to determine an energy cutoff when one or more parameters is outside of a desired range. Cutting off the energy delivery reduces the likelihood of undesired damage to the tissue. In some embodiments, the processor 114 or other processing circuitry within the generator 110 can be configured to monitor the parameters of the circuit and modulate the voltage output to not exceed a preset or predetermined electrical power (Pₘₐₓ). In some embodiments, Pₘₐₓ can be between about 5 W and about 100 W, including all sub-ranges and values therebetween. For example, Pₘₐₓ can be between about 45 W and about 55 W, or about 50 W. During an electrosurgical treatment, the impedance of the circuit may begin with a cardiac tissue impedance of about 1500 Ohms but then rise to 2000 Ohms as the biophysical characteristics of the tissue target changes. For example, the impedance can change as the energy delivery device or guidewire touches tissue, cuts through tissue, and then arrives in the blood pool. As such, the generator 110 can be configured to monitor this change in impedance and/or changes in other parameters and adjust the parameters of the voltage waveform and/or stop energy delivery. In some embodiments, the generator 110 can be configured to modify the RF output based on a characteristic (e.g., impedance, output current, temperature, etc.) of the energy delivery element 124. Some electrosurgical waveform generators may operate in constant power control modes, e.g., where the impedance of the circuit is continually measured, and the voltage of the RF waveform is adjusted to produce a fixed electrical power. For example, in an application involving a generator set to produce a constant power of 50 W, the voltage of the voltage waveform may dramatically rise to a maximum voltage, Vmax, of about 1000 to 3000 V (peak-to-peak) or greater to produce the constant power of 50 W with increases in tissue impedance. This can present complications, as any connected electrosurgical device must have sufficient electrical insulation to protect against dielectric breakdown or current leakages, which can be hazardous to the patient or operator at high voltages. High voltages associated with these power-controlled electrosurgical generators may produce sparking and fulguration at the tip of the guidewire, which can produce significant gas bubbles, coagulum, and char formation. For an intravascular device within the left atrial chamber of the heart, these can lead to ischemic stroke or other complications. In some embodiments, the generator is configured to vary the RF output after time. For example, the generator can provide a first output for a first period of time, a second output for a second period of time, a third output for a third period of time, and/or so on.

Given the lower operating voltages of the generator 110 and the use of a feedback control scheme that maintains power below a predetermined peak power, systems and devices described herein can be used with electrosurgical devices having smaller profiles. For example, the generator 110, by operating with lower voltages (e.g., voltages between about 150 V and about 250 V), can allow for the use of electrosurgical devices with smaller insulators or less insulating material. In particular, the ability of an insulated wire to resist dielectric breakdown is directly related to the thickness of the insulator. As such, with lower voltages, electrosurgical wires with thinner insulative coating can be used. In some embodiments, the coating thickness of the guidewires can be negligible compared to the diameter of the core of the guidewires, which can result in improved mechanical characteristics of the energy delivery element 124. Furthermore, since the insulator is thin, it may be applied to the guidewire core via low-cost manufacturing techniques such as dip or spray coating, or by extrusion.

In some embodiments, the generator 110 can be configured to modulate the voltage output based on a temperature of a distal tip of the energy delivery element 124. As noted above, using a RF powered guidewire or needle to create an atrial-septal defect (e.g., for transseptal access) can cause thromboembolic risk by generating inadvertent char and/or coagulum. Char and coagulum are created when tissue reaches temperatures above a threshold, which causes protein denaturation, dehydration, and thrombogenic cascade. Maintaining the tip temperature below that threshold would prevent the formation of char and coagulum, and thus avoid thromboembolic risk to the patient. Therefore, in some embodiments, the generator 110 can be configured to deliver a voltage output to the energy delivery element 124 until a target set point temperature or range is reached. In some embodiments, the target set point temperature or range can be between about 55 and about 80 degrees Celsius. In operation, the RF output from the generator can be initiated by a user, e.g., by actuating an actuator (e.g., button or slider which may be located on the electrosurgical interface 122 described below), when the guidewire is located at the desired area of tissue contact. The generator 110 can then deliver current to reach the target set point temperature. By avoiding temperatures above about 80 degrees Celsius, the incidence of char and coagulum formation can be reduced or avoided.

FIG. 1B provides a more detailed view of the generator 110. As seen if FIG. 1B, the memory 112 can store processor-executable instructions that, when executed by the processor 114, cause the processor to implement signal acquisition 114a, signal processing 114b, electrical parameter determination 114c, state monitoring 114d, and energy adjustment 114e. As described below, the processor 114 can be configured to monitor the parameters associated with the generator 110 which can then be used to determine additional parameters, monitor the state of one or more component of the electrosurgical system 100, and/or adjust the functionality of the generator 110.

The signal acquisition 114a includes measuring and/or receiving signals (e.g., sensor data, etc.) associated with the energy delivered by the generator 110 to the electrosurgical device 120. For example, the signals can be associated with voltage, current, and/or the like. In some embodiments, the generator 110 can include at least one sensor for measuring the signals. In some embodiments, the at least one sensor can be in series with the output of the generator. For example, the generator 110 can include a voltmeter, ammeter, multimeter, and/or the like. In some embodiments, the generator 110 can be operatively coupled to a sensor configured to measure the signals and send the signals to the generator 110. In some embodiments, measuring the signals can be continuous, periodic, and/or sporadic. In some embodiments, each of the signals can be sampled simultaneously. In some embodiments, an analog-to-digital converter can be used to sample the signals. In some embodiments, the signals can be sampled with a buffer size of approximately 200 samples at a sub-Nyquist frequency of 100 kHz providing an intermediate frequency of approximately 20 kHz. In some embodiments, the signals can be processed above the Nyquist frequency. In some embodiments, the sampling frequency is about 10 MHz

The signal processing 114b includes processing the signals acquired during signal acquisition 114a. Processing the signals can include calculating a magnitude of the signals, determining complex values (e.g., phasor value) for the signals, and/or the like. The complex values can include a real part and an imaginary part of the signal. For example, determining the magnitude can include determining an RMS value of the signals and/or the magnitude of the complex values. Determining the complex values can include using a Goertzel algorithm, Fourier transform (e.g., fast Fourier Transform), phase tracking algorithm, and/or the like. In some embodiments, processing the signals can include determining at least one of RMS voltage, RMS current, a complex representation of voltage, a complex representation of current, and/or the like. In some embodiments, the signal processing 114b can include determining a phase angle between two signals of the signals acquired during signal acquisition 114a. For example, the phase angle between a voltage signal and current signal can be determined. In some embodiments, determining the phase angle can be based on complex impedance.

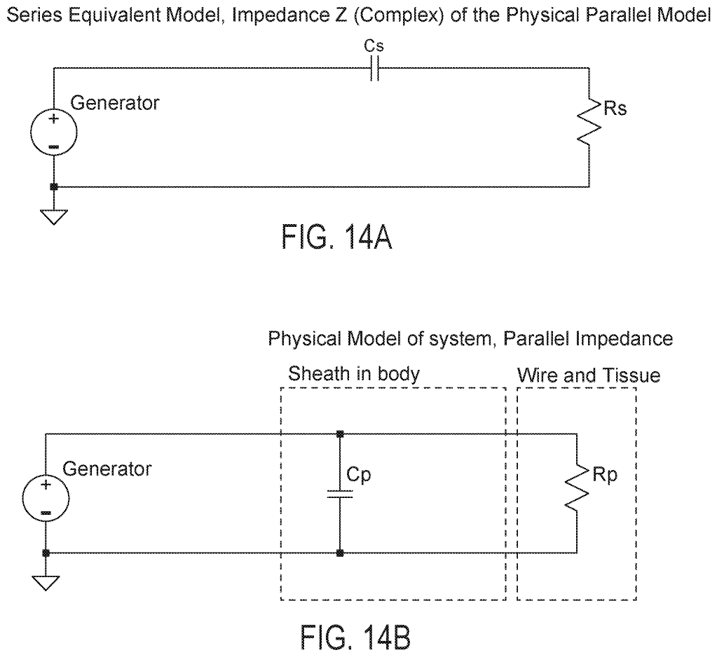

The electrical parameter determination 114c can include using the signals acquired during signal acquisition 114a and processed during signal processing 114b to determine one or more electrical parameter associated with the electrosurgical system 100. In some embodiments, at least one of the electrical parameters can be associated with a parallel approximation of the electrosurgical system 100 as further described in reference to FIGS. 14A-14B. In some embodiments, the electrical parameters can include at least one of complex impedance, impedance magnitude, tissue impedance, reactance, capacitance of the sheath, apparent power, real power, power factor, total energy delivered, current, and/or the like.

The state monitoring 114d can include using the electrical parameters determined during electrical parameter determination 114c to determine one or more state associated with the electrosurgical device 120 and/or the tissue during operation. The states can be associated with a position of one or more component of the electrosurgical device 120 such as the energy delivery element 124 and/or the sheath. The states can be associated with the energy delivery element 124 engaging tissue and the states can be associated with whether the tissue has been engaged, partially punctured, successfully punctured, and/or unsuccessfully punctured. In some embodiments, the state can include whether the energy delivery element 124 is disposed in or extending beyond the sheath 126, whether the energy delivery element 124 and/or the sheath 126 is moving toward or away from the tissue, whether the energy delivery element 124 has engaged the tissue, whether the sheath 126 has engaged the tissue, whether the energy delivery element 124 has partially and/or fully punctured the tissue, whether the energy delivery element 124 has engaged heart wall, whether the energy delivery element 124 has entered the blood pool in the left atrium, and/or the like. The different states are further described in reference to FIG. 10 below. In some embodiments, determining the one or more states can be based on the values, rate of change, changes, and/or the like of the electrical parameters. For example, an impedance value and/or a voltage value can indicate if the energy delivery element 124 has engaged the tissue and/or punctured the tissue.

The energy adjustment 144e includes adjusting the energy delivered by the generator 110 to the electrosurgical device 120 based on at least one of the state and/or the electrical parameters. Adjusting the energy delivery can include decreasing, increase, or adjusting a set voltage, set current, set power, and/or the like. In some embodiments, stopping energy delivery after the energy delivery element 124 enters the blood pool beyond the septum provide protection of non-fossa tissue in the atrium. Additionally, stopping energy delivery after the energy delivery element 124 enters the blood pool beyond the septum can reduce coagulum on the tip of the energy delivery element 124 and can reduce the formation of microbubbles (e.g., gaseous and particulate) associated with undesirable heating of the blood.

The energy adjustment 144e can include stopping energy delivery if the energy delivery element 124 is extended out of the sheath 126, but has not yet engaged the tissue, to decrease the likelihood of coagulum build up prior to tissue engagement and traversal. In some embodiments, the generator 110 can be configured to generate an alert to a user that indicates that a predefined condition (e.g., a predefined threshold, a predefined range of values, a predefined change in a parameter) associated with the energy delivery element 124 has occurred. In some embodiments, the predefined condition can be associated with a predefined state of the energy delivery element 124. In some embodiments, the energy adjustment 144e can include one or more shut off thresholds or other conditions where energy delivery is terminated. For example, the shut off threshold or condition can be associated with an impedance value, an increase in impedance exceeding a value then dropping below the value, an increase in real power increase above a predetermined power threshold, etc. Additional thresholds and/or other criteria can be used to alter the energy generated by the generator 110.

The electrosurgical device or assembly 120 can include an electrosurgical interface 122, an energy delivery element 124, and a sheath 126. While the electrosurgical device 120 is described as a single device, it can be appreciated that each of the electrosurgical interface 122, energy delivery element 124, and sheath 126 can be implemented as separate devices and/or components of two or more devices.

The electrosurgical interface 122 establishes electrical connectivity between the generator 110 and the energy delivery element 124. The electrosurgical interface 122 can be coupled to, coupleable with, or include a cable that connects to the generator 110 and an interface for receiving the energy delivery element 124. The interface 122 can include an electrical coupling element that is electrically connected to the generator 110 (e.g., via the cable). In some embodiments, the electrosurgical interface 122 establishes electrical connectivity between the generator 110 and the energy delivery element 124 while allowing for slidable translation of the energy delivery element 124 therethrough. In an embodiment, the electrical coupling element is an electrode and the electrosurgical interface 122 includes a layer of conductive fluid in electrical contact with the electrode. The energy delivery element 124 can then be received within the conductive fluid and be electrically coupled to the electrode via the conductive fluid. The energy delivery element 124 is slidable within the conductive fluid, without loss in the electrical coupling with the electrode. In some embodiments, electrical coupling between the energy delivery element 124 and the electrosurgical interface 122 can include direct coupling, fluid coupling, induction coupling, or the like. In some embodiments, the interface 122 can be electrically coupled to the end of the energy delivery element 124.

In some embodiments, the electrosurgical device 120 can include a button, slider, or other actuation device for establishing the electrical connection between the generator 110 and the electrosurgical interface 122. For example, a button or slider can be provided on the electrosurgical device 120, e.g., near where a user may be manipulating the energy delivery element 124 and/or other components of the electrosurgical device 120, and the button can be pressed, or the slider can be slid to establish electrical connection between the generator 110 and the electrode of the electrosurgical interface 122. Alternatively, or additionally, a button, slider, or other actuation device can be actuated to send a signal to the generator 110, e.g., via a wired or wireless connection to the generator 110. In some embodiments, the signal can be an activation (on or off) signal. In some embodiments, the signal can trigger the generator 110 to send RF energy (e.g., a pulse waveform) to the electrosurgical device 120. In some embodiments, the signal can be a voltage signal, a current signal, or an impedance signal, which can be communicated to the generator 110 and the generator 110, in response to receiving the signal, can deliver a RF waveform to the electrosurgical device 120. In some embodiments, the electrosurgical interface 122 and the generator 110 are configured to communicate to identify the type and/or information regarding the electrosurgical interface 122, generator 110, and/or the energy delivery element 124. For example, the generator 110 can determine if the energy delivery element 124 has been previously used for a procedure. In some embodiments, the generator 110 may be configured to prevent reuse of the energy delivery element 124 to decrease the chance of contamination.

In some embodiments, the electrosurgical interface 122 can include a cutting feature to remove portions of an insulating jacket of the energy delivery element 124 to electrically couple to the energy delivery element 124. In some embodiments, the electrosurgical interface 122 can include a button, that when activated, operates a cutting blade to expose a conductive portion of the energy delivery element 124.

The energy delivery element 124 can include an electrode or other conductive element for applying energy to a tissue structure. In embodiments, the energy delivery element 124 is a wire (e.g., guidewire) that includes a distal conductive region that is used to apply energy to and thereby puncture through tissue structures. In some embodiments, the energy delivery element 124 includes a distal region that can transition between different configurations or shapes, e.g., a curved configuration vs. a straight configuration. In some embodiments, the energy delivery element 124 can have a shape memory region that automatically assumes a preset shape or configuration when unsheathed by more than a certain amount (e.g., from a sheath 126, such as described below). For example, the energy delivery element 124 can have a shape memory region that automatically curves or assumes a curved shape or atraumatic configuration (e.g., when curved, a curved portion becomes the most distal portion of the energy delivery element 124 thus making it less sharp and likely to damage off-target tissue). Alternately or additionally, the guidewire can include a spring-tempered stainless steel portion that can return to a predetermined shape, when released from a constraining sheath (such as the dilator discussed below). In some embodiments, the energy delivery element 124 can include a coiled structure that is at least partially coated, e.g., with an insulating layer. In some embodiments, the energy delivery element 124 can be formed of metallic and polymer materials. In some embodiments, the energy delivery element 124 can include more than one coiled structures. For example, a coated coiled structure and an uncoated coiled structure. In some embodiments, the energy delivery element 124 can include an insulating coating near the tip. In some embodiments, the energy delivery element 124 can include material near the distal tip that provides radiopacity. echogenicity, and/or insulation (e.g., tantalum, tungsten, etc.).

In some embodiments, the energy delivery element 124 can have a large active electrode region or a long electrode, with a large conductive surface area, e.g., a conductive region that is greater than about 1 to 2 mm, greater than about 1 cm, greater than 2 cm, greater than 3 cm, greater than 4 cm, greater than 5 cm, or greater than 10 cm, or between about 1 cm and about 100 cm, including all values and sub-ranges therebetween, depending in part on the diameter of the electrode. The active electrode region can include the distal conductive tip of the energy delivery element 124 and a conductive outer portion of the energy delivery element 124, e.g., a conductive outer coil, plating, etc. The larger active electrode region can provide cooling to the tip, as the conductive/uninsulated region of the guidewire can wick or conduct away heat. The larger active electrode region can also reduce current density at a distal tip of the energy delivery element 124, thereby reducing the risk of forming undesired and/or inadvertent lesions at locations in the heart other than the septum. The larger active electrode region can also enable measurements of voltage, current, and/or other parameters with higher signal-to-noise ratios, as further described herein.

In some embodiments, the energy delivery element 124 can include at least one sensor. The at least one sensor can be located in the tip of the guidewire, near the tip of the guide, in a distal portion, in a proximal portion, and/or the like. As used herein, proximal refers to the portion of a device or component closest to the surgeon and distal refers to portions closer to the patient anatomy. The sensor can be configured to measure a characteristic of the guidewire. For example, the sensor can be configured to measure temperature, current density, pressure, and/or the like. In some embodiments, the sensor can be used for locating the tip in an electroanatomic mapping system allowing the guidewire to be located in a cardiac space. In some embodiments, the sensor can be a temperature sensor such as a thermistor or thermocouple. In some embodiments, the sensor can be a bi-metal thermocouple, e.g., where there is a weld between an outer coil wire and one or more core wires. The proximal joint of the coil and core wire(s) can serve as a bi-metal thermocouple. In some embodiments, the sensor can be a thermistor that is integrated into a coil of the guidewire. The sensor can be used to provide feedback to decrease or switch off power in response to an out-of-range reading, thus improving the safety of the procedure.

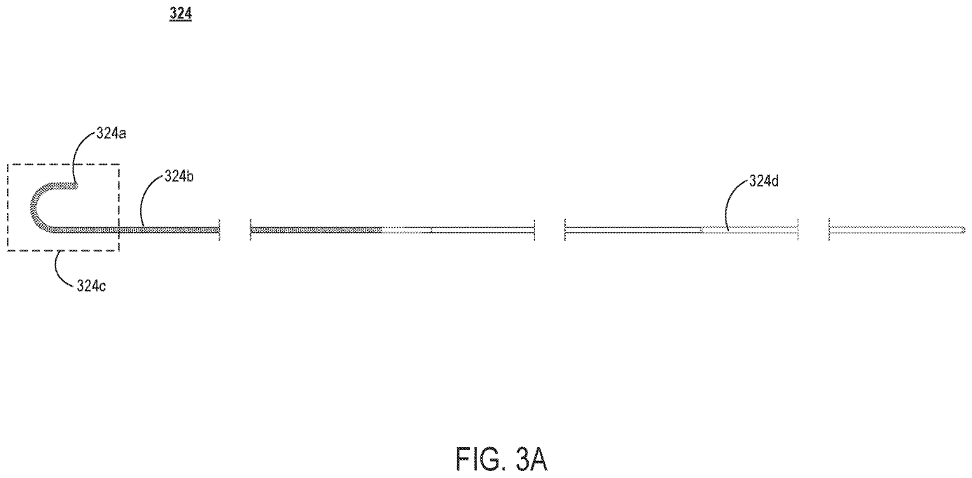

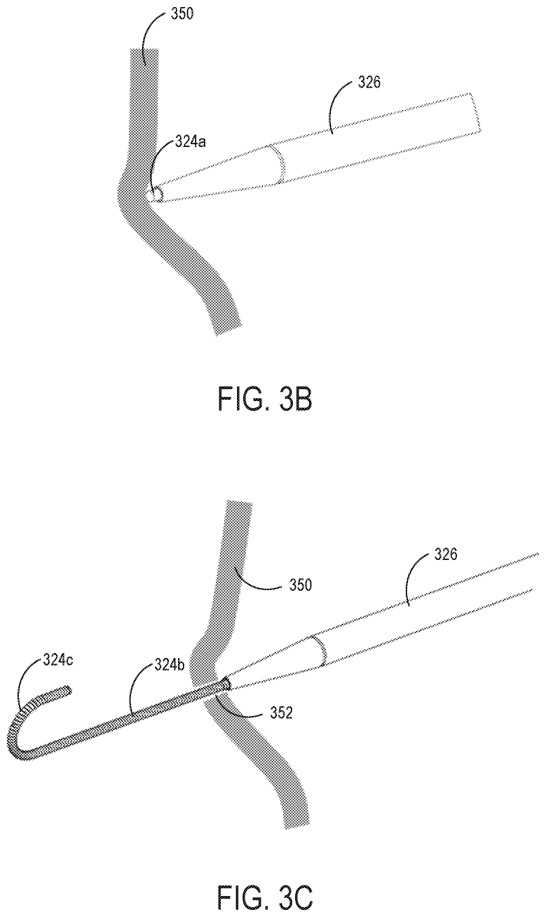

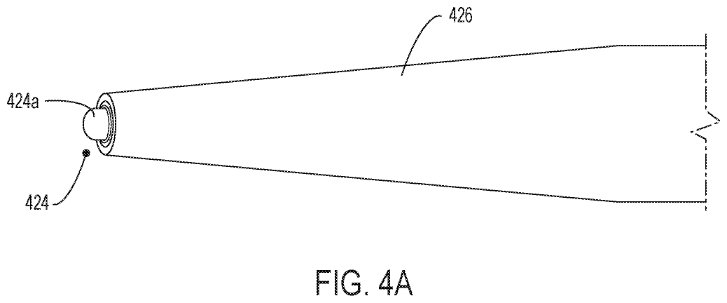

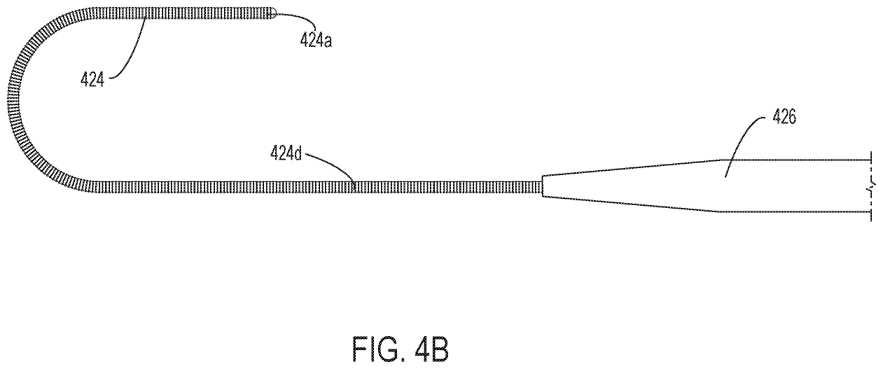

In some embodiments, the energy delivery element 124 can have a proximal length or portion that is coated with an insulator and a distal length or portion that is not. The coated portion can be grasped or manipulated by an operator (e.g., surgeon) during an electrosurgical procedure. In some embodiments, the energy delivery element 124 can be formed of materials that allow a surgeon to quickly identify or recognize the energy delivery element 124. For example, the energy delivery element 124 can have a two-tone design that includes a metallic distal colored portion and a non-conductive proximal colored portion. The metallic portion can extend toward a set point along the energy delivery element 124 (e.g., a midpoint), and acts as a contiguous conductor from the set point to the distal end of the guidewire. The metallic portion can include a metallic coating or other conductive material that covers manufacturing artifacts from welding, heat setting, or shaping, resulting in a consistent, smooth surface finish. In some embodiments, the metallic portion can include plated gold over stainless steel or another base material (e.g., tungsten). The proximal portion can have a polymer coating or other insulating material that insulates the energy delivery element 124. The polymer can be extruded, reflowed, or applied by coating. Suitable examples of such insulative materials include polytetrafluoroethylene (PTFE), polyimide, and nylon. The metallic portion and the proximal non-conductive portion can have the same or different lengths. Further details of example electrosurgical guidewires are described below with reference to FIGS. 3A-4C.

When used with the electrosurgical interface 122, the conductive portion of the energy delivery element 124 can be coupled to the electrosurgical interface 122 such that energy can be transferred via a conductive core of the energy delivery element 124 to the distal tip of the energy delivery element 124. In some embodiments, the energy delivery element 124 can include stainless steel to conduct energy (e.g., stainless steel core, stainless steel outer coil, etc.). In some embodiments, materials such as gold, platinum, and/or other highly conductive materials can be used to form and/or coat portions of the energy delivery element 124 to improve heat transfer and lower the operating temperature of the tip of the energy delivery element 124 during surgical procedures. For example, the energy delivery element 124 can be formed of such materials and/or coated or plated with such materials. Optionally, materials such as tungsten, tantalum, and/or the like can be incorporated into the energy delivery element 124 for their radiopacity. In an embodiment, the energy delivery element 124 is a conductive metallic wire can have a coiled design with a large surface area and is plated with a good thermal conductor such as gold, to improve heat transfer and lower the operating temperature of the tip. This in turn can reduce the risk of char or thrombus formation due to excessive temperatures.

A sheath 126 (e.g., insulating shaft) can be used together with a guidewire or other energy delivery element 124. The sheath 126 comprises a cannula with a lumen having an inner diameter sized to receive the energy delivery element 124 and allow for sliding of the energy delivery element 124 along the axis of the sheath. The sheath 126 can provide support to the guidewire during advancement of the guidewire through patient anatomy. In some embodiments, the sheath 126 can be configured to constrain or shape the energy delivery element 124. For example, as described above, in some embodiments, the energy delivery element 124 can have a distal tip that is configured to transition between a curved configuration and a straightened or straight configuration. The energy delivery element 124 tip can be formed of shape memory or spring-biased material and that is straight when constrained and curved when not constrained by an outer sheath (e.g., sheath 126). This can be desirable as the guidewire then has an atraumatic shape that can avoid accidental injury to nearby patient anatomy. The sheath 126 can then be used to constrain the energy delivery element 124 to a straight configuration, such that the tip of the energy delivery element 124 can contact and perforate through a tissue structure as it is advanced a first distance distally along the length of the sheath but curves as it is advanced to further distances where it would be more likely to encounter off-target anatomy. Further details of such are described with reference to FIGS. 3A-4C. In some embodiments, the sheath 126 can include or be used with a dilator. After the energy delivery element 124 forms the perforation or opening in the tissue structure, the dilator can be advanced to dilate the opening, e.g., to facilitate delivery of secondary therapeutic devices such as an ablation catheter, sheath, or other medical device. In some embodiments, the electrosurgical system 100 includes a dilator without a sheath.

The energy delivery element 124 can be designed to have universal compatibility with multiple types of sheaths, dilators, and/or other devices. In some embodiments, the energy delivery element 124 can be used with multiple types of sheaths 126. As such, a surgeon or medical practitioner can select an appropriate sheath to use during a particular operation, without requiring any specific adaptation of the system for use with the selected sheath.

In some embodiments, the guidewire or other energy delivery element 124 can include an insulating collar, disposed near a distal end of the energy delivery element 124. In some embodiments, the insulating collar is optional. The insulating collar can have a length of between about 2 mm and about 10 mm, including all sub-ranges and values therebetween. The insulating collar surrounds a conductive portion of the energy delivery element 124 near the distal end. In some embodiments, the collar can be disposed between about 0.5 mm to about 3 mm from the distal end of the energy delivery element 124, including all sub-ranges and values therebetween. In operation, as the energy delivery element 124 is extended distally out of the sheath, the conductive tip of the guidewire is exposed. Further extension of the energy delivery element 124 out of the sheath would then expose the insulating collar of the energy delivery element 124. The insulating collar can act as an extension of the insulative sheath such that the total surface area of the exposed conductive portion of the energy delivery element 124 remains small, thereby maintaining higher current density levels near the distal tip of the energy delivery element 124. This ensures that the distal tip of the energy delivery element 124 has sufficient energy to penetrate through the septum, when the energy delivery element 124 is extended a short distance out of the sheath. As the guidewire is extended even further out from the sheath and is inserted into the blood pool beyond the septum (e.g., the left atrium), additional conductive portions of the energy delivery element 124 then become exposed, thereby reducing the current density at the distal tip of the energy delivery element 124. This then reduces the risk that the energy delivery element 124, when disposed beyond the septum, may contact and inadvertently form lesions in the heart wall (e.g., myocardium). Further details of the properties and operation of the guidewire with an insulating collar are described with reference to FIGS. 4A-4C.

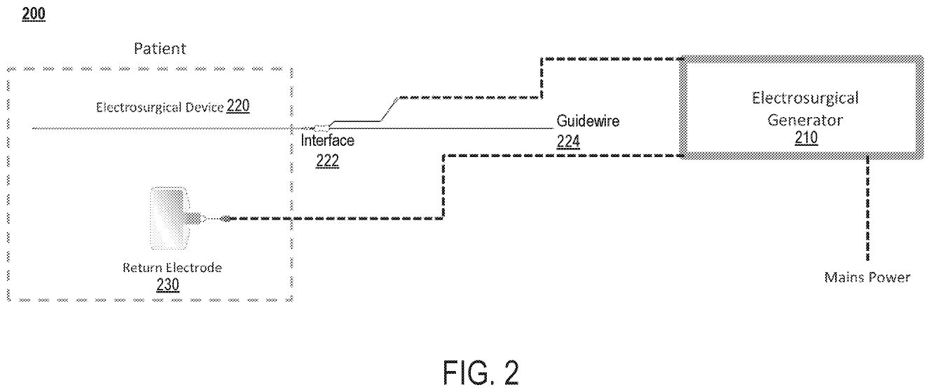

FIG. 2 is a schematic diagram of an electrosurgical system 200, according to embodiments. The electrosurgical system 200 can be structurally and/or functionally similar to other electrosurgical systems described herein, including, for example, electrosurgical system 100. For example, the electrosurgical system 200 can include an electrosurgical device 220 (e.g., structurally and/or functionally similar to electrosurgical device 120), an electrosurgical generator 210 (e.g., structurally and/or functionally similar to generator 110), and a return electrode 230 (e.g., structurally and/or functionally similar to return electrode 130).

Similar to the electrosurgical system 100, the electrosurgical system 200 can be configured to puncture and cross thin tissue structures for biomedical applications. The system 200 includes the generator 210, an interface 222, a removable guidewire 224 (e.g., an example of an energy delivery element 124) that delivers electrosurgical energy to a therapeutic target (e.g., a tissue structure within the patient), and the return electrode 230 attached to the body of the patient. In the illustrated embodiment, interface 222 is electrically coupled to a medial portion of guidewire 224 by an electrical contact affixed to the fluid lumen of an intravascular sheath or a dilator. In another embodiment, interface 222 may be electrically coupled to a proximal end of guidewire 224 by a mechanical connection.

In use, the electrosurgical energy flows from the generator 210, into the patient and to the exposed tip of the guidewire 224 that is in contact with the target tissue. The circuit is completed by the return electrode 230 attached to the body of the patient, which may be located on the torso or extremity such as the patient's leg. In some embodiments, the electrosurgical generator 210 connected to the electrosurgical device 220 can generate an oscillating sinusoidal RF waveform having a frequency of approximately 470 kHz and a peak voltage of approximately 285 V RMS. In some embodiments, the generator 210 can operate in constant power mode with a setting of between about 5 W to about 50 W.

In some embodiments, similar to the generator 110, the generator 210 can implement a feedback loop control scheme, whereby the generator 210 adjusts one or more parameters of the RF waveform based on a measured impedance or other characteristic of the circuit. The generator 210 can include circuitry for monitoring the electrical impedance of the circuit going from the generator 210 to the electrosurgical device 220 and to the return electrode 230 and back to the generator 210. The generator 210 (e.g., via an onboard processor or circuitry) can calculate the instantaneous power as provided by the equation P=I*V, where P is power, I is current, and V is voltage. The generator 210 can be programmed to have a predetermined peak voltage (Vₚₑₐₖ) and a peak power (Pₚₑₐₖ), and during electrosurgery, the peak voltage of the voltage waveform can be set to Vₚₑₐₖ unless the calculated instantaneous impedance value or current value is exceeded, in which case V is reduced so that the power remains below or equal to Pₘₐₓ.

In some embodiments, similar to the generator 110, the generator 210 can monitor current and/or voltage associated with delivering energy via the electrosurgical device 220 to the patient. The monitored current and/or voltage can be used to calculate or determine other parameters that can be monitored to determine if an energy cut-off state is present. The generator 210 can determine that it is desirable to turn off the energy delivery based on at least one of current, voltage, impedance, power (e.g., real power), capacitance, and/or the like.

Other examples of suitable electrosurgical devices, including electrosurgical interfaces and energy delivery elements, are described in International Patent Application No. PCT/US2023/086043, filed Dec. 27, 2023, the disclosure of which is incorporated herein by reference.

### Energy Delivery Element or Guidewire

FIGS. 3A-3B depict a guidewire 324 of an electrosurgical device (e.g., electrosurgical device 120, 220), according to an embodiment. FIG. 3A provides a side view the guidewire 324 with break points to better show details of the various components of the guidewire 324. FIGS. 3B and 3C depict perspective views of a guidewire 324 within a dilator 326 forming an opening and pushing through tissue 350. The guidewire 324 may be functionally and/or structurally similar to the energy delivery element 124 of FIG. 1A.

As depicted in FIG. 3A, an embodiment of the guidewire 324 includes a tip 324a, a distal curved portion 324c, a distal segment 324b, and proximal segment 324d. The guidewire 324 is formed of stainless steel with a tapered core wire and a distal coil welded at the tip 324a and a proximal termination (e.g., the distal end of the proximal segment 324d). Optionally, the core wire may be formed of nitinol. The guidewire 324 is formed of biocompatible and sterile materials.

The distal segment 324b and/or proximal segment 324d can conduct energy when in use (e.g., from an electrosurgical interface). The distal segment 324b may be conductive along its length. The proximal segment 324d may have an exposed conductive region that is configured to electrically couple with an electrode of an electrosurgical interface. When energized, the proximal segment 324d conducts this energy to the distal tip 324a of the guidewire. In some embodiments, the proximal segment 324d may be partially or wholly insulated so that it may be handled while in use. For example, a portion of the proximal segment 324d may be covered with an insulative layer. The insulation can prevent electrical current from flowing through the wire and into the hands of a physician. The insulative coating may be, for example, polytetrafluoroethylene, perfluoro alkoxy alkane, fluorinated ethylene propylene, polyether ether ketone (PEEK), polyimide, epoxy, ceramic, nylon, and/or a composite of insulating materials. The proximal segment 324d is stiff to facilitate exchange of large bore sheaths through tortuous patient anatomy. The distal segment 324b and/or the proximal segment 324d (or portions thereof) can have a gold plating applied over a stainless-steel core to improve conductivity, including heat conductivity. Such conductivity may be beneficial for conductive heat away from a distal tip of the guidewire, e.g., to prevent overheating at the distal tip of the guidewire. In some embodiment, the core of the guidewire 324 may be coated in tungsten or other conductive material.

The distal segment 324b can be less stiff than the proximal segment 324d. In some embodiments, the distal segment 324b includes a non-conductive or insulating coating over a portion of it or the distal segment 327b may be uninsulated. For example, as described with reference to FIG. 1A, the distal segment 324b may have an insulating collar that surrounds a short portion of the guidewire near or adjacent to the distal tip of the guidewire. In some embodiments, the distal collar may be between about 2 mm and about 10 mm in length, including all sub-ranges and values therebetween. The distal end of the distal segment 324b includes a curved portion 324c. The curved portion 324c is formed of a shape memory material. The curved portion 324c may be a J-shape, as in FIG. 3A, but other shapes are possible including a straight shape and/or other curved or spiral shapes (e.g., a pigtail, a coil, etc.). The J-shape of FIG. 3A is atraumatic as to no cause damage to patient anatomy while being navigated therethrough. The guidewire 324 can remain in its J-shape when unconstrained and being navigated through patient anatomy to a target tissue. For applying energy to tissue, however, the guidewire 324 can be constrained to a straighter configuration, e.g., so that the distal tip 324a of the guidewire is positioned to apply energy to and puncture through tissue. For example, when the guidewire 324 is retracted within a sheath or dilator (e.g., the sheath 126), the guidewire 324 can thereby be constrained to a straight or substantially straight configuration. The tip 324a is a conductive and/or non-insulated portion of the distal segment 324b. The tip 324a can be energized to deliver energy (e.g., RF energy) to tissue to create a perforation or opening; for example, by actuating the electrosurgical interface 122 to transfer energy from the generator 110 to an exposed region of the wire positioned therein. As discussed above, the guidewire 324 can again take a J-shape if extended beyond a perforation in the target tissue.

FIGS. 3B and 3C show the guidewire 324 in use. The guidewire 324 and the sheath 126 can be advanced through patient vasculature to a target site. In FIG. 3B, the guidewire 324 is sheathed in a dilator 326 (e.g., functionally and/or structurally similar to the sheath 126 of FIG. 1A and/or other sheaths and dilators described herein) such that only the tip 324a protrudes from the dilator 326. In such a configuration, the tip 324a is located in line (or approximately in line) with a longitudinal axis of the dilator 326. In some applications (e.g., when crossing the septum), the tip 324a can press against the tissue 350, as shown in FIG. 3B. This can be part of tenting the tissue. Upon actuation, RF energy is transmitted from the generator to the tip 324a and heats the tissue 350 at the point of contact. Heating the tissue 350 results in dehydration, protein denaturing, vaporization, and a loss of mechanical integrity of the tissue, thus creating a puncture. The RF energy can then be terminated, and the unenergized guidewire 324 can be advanced through the puncture. The dilator 326 may then be advanced over the guidewire 324, through the puncture and into the left atrium. In some embodiments, energy input can be terminated if a puncture attempted is failed.

In FIG. 3C, a puncture 352 has been formed in the tissue 350 and a portion of the distal segment of the guidewire 324 has been advanced through the puncture 352. As can be seen in FIG. 3C, the dilator can have a conical to tapered tip such that when the dilator 326 is pushed farther through the puncture 352, it expands or dilates the puncture. A secondary therapeutic device (e.g., ablation catheter, sheath, etc.) may be advanced through expanded puncture, optionally over the guidewire 324 after removal of the dilator 326. Thus, the guidewire 324 acts as both an energy delivery device and a mechanism for guiding further surgical instrumentation or medical devices.

FIGS. 4A-4C depict yet another example of a guidewire 424, according to embodiments. The guidewire 424 can be structurally and/or functionally similar to other energy delivery elements or guidewires described herein, including, for example, energy delivery element 124, guidewire 324, etc. As such, the guidewire 424 can include components that are similar to those components of other guidewires described herein. For example, the guidewire 424 can include a tip 424a and an electrically conductive portion 424d. The guidewire 424 is configured to be disposed within a dilator or sheath 426, which can be functionally and/or structurally similar to other dilators and/or sheaths described herein (e.g., sheath 126, 326, etc.).

FIG. 4A depicts the guidewire 424 extended a short distance out of an outer sheath or dilator 426. For example, the guidewire can be extended a distance of between about 0.5 mm and about 5 mm, including all values and sub-ranges therebetween. As described above, e.g., with reference to FIG. 3B, the guidewire can be extended a short distance out of a dilator 426 to expose the conductive tip 424a of the guidewire. The conductive tip 424a can then be used to deliver energy to perforate through tissue. When extended these short distances out of the dilator 426, the guidewire has a small conductive surface area that is exposed. This small conductive surface area can allow current density to be concentrated at the tip 424a of the guidewire, thereby enabling the tip to generate sufficient energy to perforate through a tissue wall, such as, for example, a septum.

As shown in FIG. 4B, the guidewire can be extended a further distance beyond the distal end of the dilator 426. As described above with reference to FIG. 3C, after the guidewire punctures or perforates through target tissue (e.g., a septum wall), the guidewire can be extended beyond the tissue. In some embodiments, the guidewire can be used as a delivery catheter for additional instruments. For example, the guidewire can be used to perform transseptal crossing, and then be used to guide additional instruments (e.g., electrosurgical or treatment devices, catheters, etc.) into the left atrium of the heart. In some embodiments, the guidewire can be extended a second distance of greater than about 1 cm, great than 5 cm, or great than 10 cm from the distal tip of the dilator.

When the guidewire is extended out this greater distance, as shown in FIG. 4B, the guidewire can be configured to assume an atraumatic shape, e.g., such as a J-shape, pigtail shape, etc. The atraumatic shape can be configured to reduce injury to nearby tissue structures. When the guidewire is extended out this greater distance, a distal conductive surface or portion 424d of the guidewire is also exposed. This distal conductive portion 424d can be the exposed surface of a metallic coil or other electrically conductive material. The coil can be electrically coupled to a core 424c of the guidewire, as depicted in FIG. 4C. Therefore, energy delivered to the tip can be spread across the tip and the additional exposed conductive surface 424d. The tip and the exposed conductive surface 424d can collectively function as a long electrode or larger conductive region. This greater conductive surface area causes the current density to drop, and therefore further prevents and/or reduces the possible formation of lesions in nearby tissue structures. For example, when used in a transseptal crossing procedure, the greater exposed conductive surface area of the guidewire can reduce the likelihood of injuring the heart wall, as described herein.

While the additional conductive surface 424d is shown as the exposed surface of a coil in FIGS. 4B-4C, it can be appreciated that other conductive surfaces can be used to increase the total conductive surface area of the guidewire. For example, the conductive surface 424d can also be formed of an insulative coating that includes cutouts or patterns formed therein that exposed conductive elements underneath.

FIG. 4C provides a detailed cross-sectional view of the guidewire 424. As shown in FIG. 4C, the core 424c of the guidewire extends throughout a length of the guidewire. In some embodiments, the core 424c can have a proximal section that has a larger diameter than a distal section. In other words, the core 424c can have a proximal section with a larger cross-sectional area and be tapered down to a smaller cross-sectional area at the distal end. At or near a proximal end of the guidewire, the core 424c can be exposed, such that the core 424c can be placed in electrical communication with a generator (e.g., via any of the electrosurgical interfaces described herein, e.g., electrosurgical interfaces 122, 222, etc.). Alternatively, in some embodiments, the core 424c can be covered by an insulating material but include one or more openings (e.g., holes, slots, or other cut-outs) that enable transfer of electrical current from the generator to the core 424c. Moving distally from point A, the core 424c can then be covered by an insulative coating or layer 424e, such as, for example, a polymer. The insulative layer 424e can cover the core 424c until a distal segment of the guidewire, starting at point B. This distal segment can be between about 10 to about 100 cm in length, including all values and sub-ranges therebetween.

Starting at point C, the guidewire can have a conductive coil 424d on the outside. The conductive coil 424d, as described above, can be formed of a conductive material, such as a metal or metal alloy. The coil 424d can then extend distally from point C to the tip 424a of the guidewire. Alternatively, in some embodiments, the coil 424d can extend from where the insulative layer 424e terminates (i.e., point B), or substantially where the insulative layer 424e terminates, to the tip 424a of the guidewire. As described above, the coil 424d can be coupled to the distal tip 424a such that the coil 424d can form a long electrode or conductor with the tip 424a. When the coil 424d is exposed along with the tip 424a, e.g., beyond the distal end of a dilator, the coil 424d and the tip 424a can provide a larger surface area over which energy or current delivered to the guidewire can be spread out, e.g., to reduce a current density at the tip 424a. As noted above, the core 424c can taper along its length in the distal segment of the guidewire, e.g., to provide greater flexibility closer to the distal end of the guidewire.

As shown in the enlarged view of a distal end of the guidewire in FIG. 4C, the guidewire can also include a second coil 424b, which can be disposed around the core 424c near the distal tip 424a. In some embodiments, the second coil 424c can be formed of a material that is more radiopaque than the material of the core 424c or coil 424d, such that it facilitates visibility of the guidewire distal end under imaging (e.g., fluoroscopy). In some embodiments, the first coil 424c and the second coil 424b can be compression coils. In some embodiments, the second coil 424b is optional.

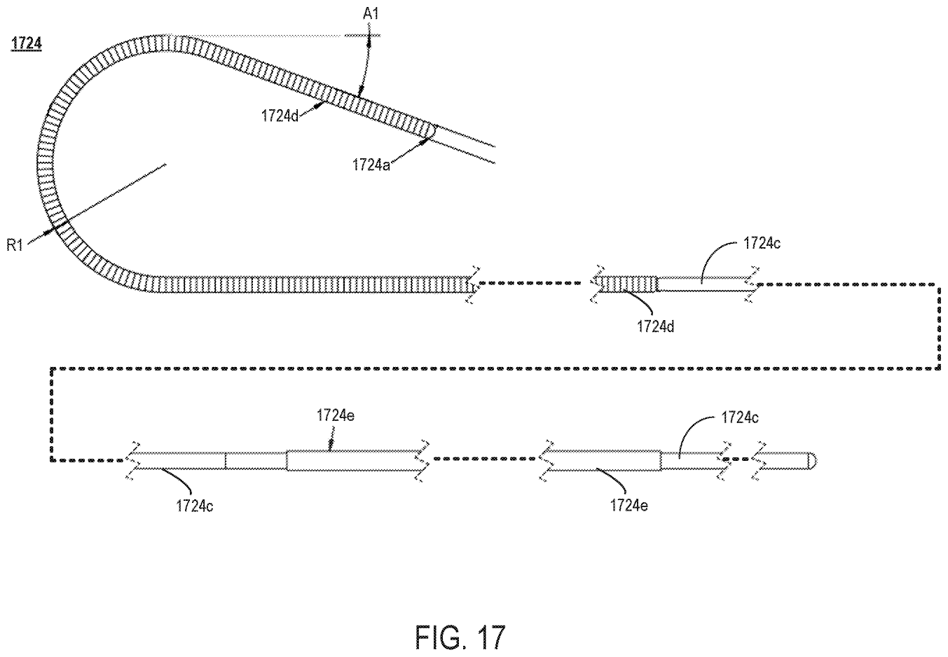

FIG. 17 depicts an alternative guidewire 1724. The guidewire 1724 can be functionally and/or structurally similar to the energy delivery element 124, the guidewire 324 of FIGS. 3A-3C, and/or the guidewire 424 of FIGS. 4A-4C. In particular, the guidewire 1724 is substantially similar to the guidewire 424 of FIGS. 4A-4C but can have a curved portion that forms a different shape. Similar to the other guidewires described herein, the guidewire 1724 include a tip 1724a, a core 1724c, a coil 1724d, and an insulative layer 1724e. The tip 1724a and the coil 1724d can collectively form an electrode that can be configured to be used to perforate tissue (e.g., septum). The electrode can be a long electrode (e.g., have a length greater than at least about 1 cm). The guidewire 1724 defines a curved portion with a radius R1. In some embodiments, the radius R1 is about 0.2 cm. Distal to the curved portion is an angled portion. In some embodiments, the angled portion defines an angle A1 relative to the distal end of the curved portion. In some embodiments, the angle A1 is between about 0 degrees and about 25 degrees. In some embodiments, the angle A1 is about 20 degrees.

As described in further detail herein, a generator (e.g., generator 110 of FIG. 1 and/or the generator 210 of FIG. 2) can be configured to implement an automatic energy shut off algorithm. The generator can be configured to monitor one or more parameters associated with a distal end of an electrosurgical device, such as, for example, the guidewires 324, 424, 1724. For example, the generator can be configured to measure a voltage, current, or other electrical parameter via a conductive distal end of the guidewire (e.g., the tip 424a, 1724a and/or the conductive coil 424d, 1724d). Given the longer conductive region (e.g., a longer electrode design) of the distal end of the guidewire, the guidewire can be designed to capture signals that have a higher (e.g., greater than 10, etc.) signal-to-noise ratio than a smaller tip electrode (e.g., a tip electrode having a length of about 1-2 mm). Because the longer conductive region would have a larger exposed length, it can minimize the effects on measured parameters such as, for example, impedance, due to changes in tissue properties (e.g., due to coagulation, vaporization, etc.), temperature, or other environmental factors occurring at a tip of the guidewire. In other words, where measurements captured by a small tip electrode may be easily affected by changes in tissue properties, temperature, or other environmental factors at the location of the electrode, such changes have a lesser effect on longer electrodes that are exposed to a larger region of tissue, blood, etc. Therefore, the longer electrode of the guidewire can enable capture of voltage, current, and/or other parameters with higher signal-to-noise ratios.

While guidewires with curvatures are depicted herein, it can be appreciated that systems, devices, and methods described herein can also be used with guidewires having a linear or straight configuration (i.e., no curvature).

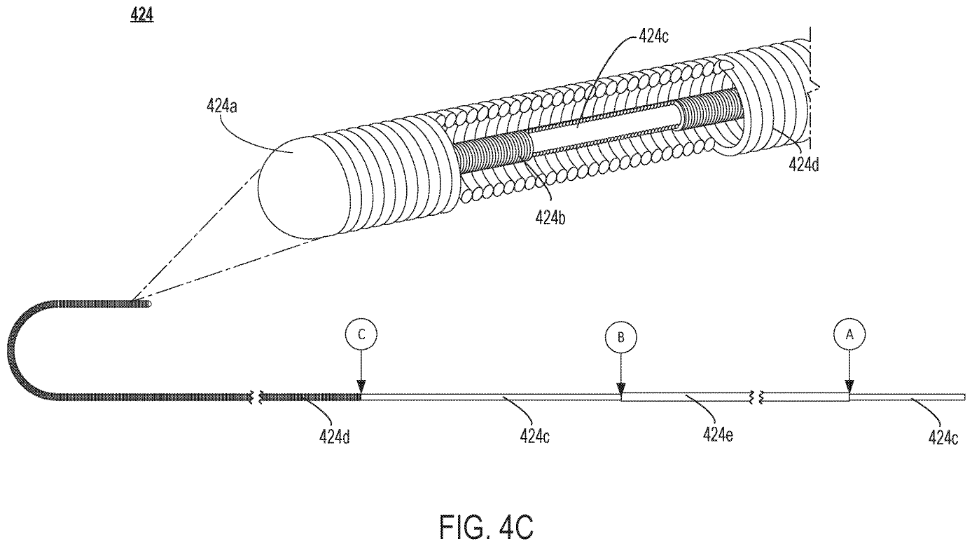

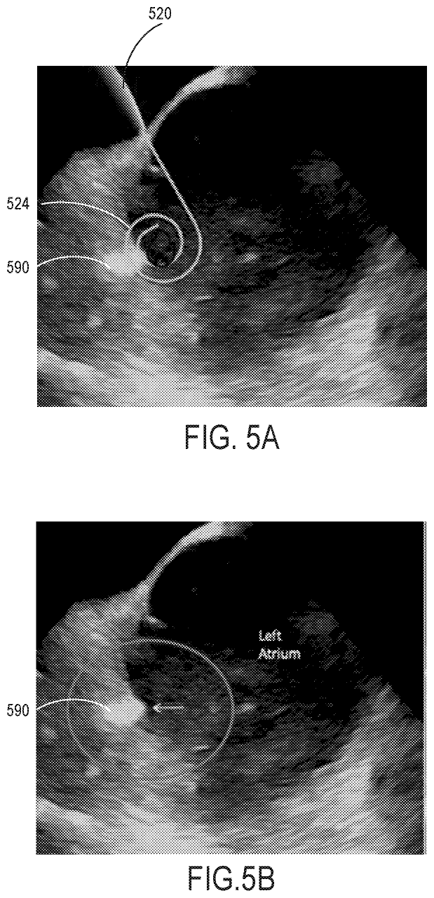

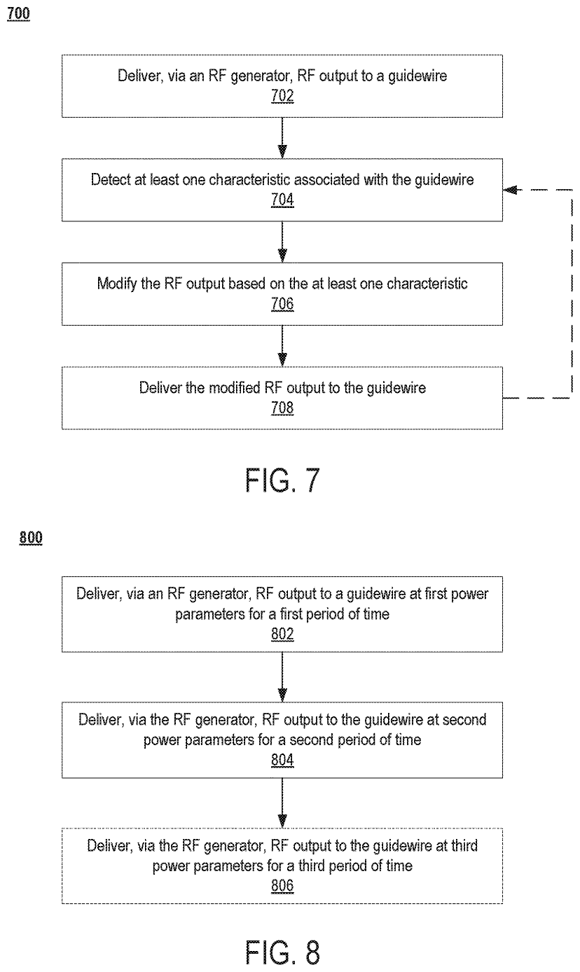

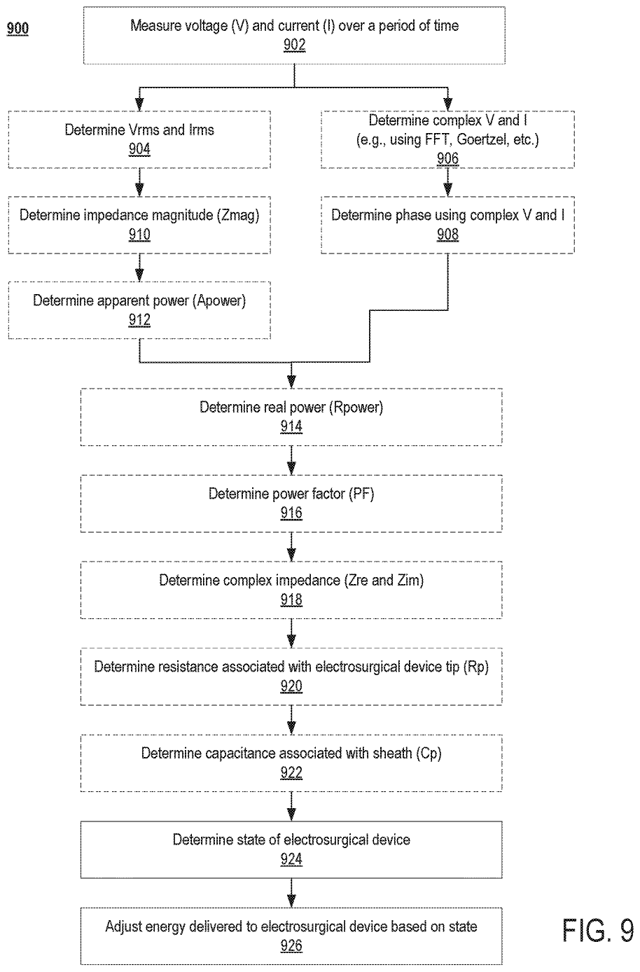

FIGS. 5A-5B depict an unintentional lesion being formed by an electrosurgical device accessing a heart. The heart depicted includes the unintentional lesion 590 in the left atrium, as seen in FIGS. 5A and 5B. FIG. 5A depicts an illustration of the lesion 590 being formed due to a guidewire of a transseptal device or other electrosurgical device 520. The guidewire of the electrosurgical device 520 may have contacted the wall of the left atrium after deploying from a sheath causing the lesion 590. The systems, devices, and methods described herein are configured to decrease the likelihood of lesions, such as lesion 590 forming. For example, the methods described in reference to FIGS. 7 and 8 below can be configured to decrease RF output when the RF output of the electrosurgical device is not desired. Similarly, the method described below in reference to FIG. 9 can reduce the likelihood of lesions by stopping RF output when the RF output of the electrosurgical device is not desired. Additionally or alternatively, the configuration and design of the guidewires or energy delivery elements, as described in the following figures, can reduce the risk of lesion formation, e.g., by having lower current density as the guidewires are extended distally out from a dilator or outer sheath.

In some embodiments, the guidewires or energy delivery elements of the electrosurgical systems described herein can be configured to have lower current density when they are extended distally beyond a septum, while still maintaining higher current density when puncturing through the septum. In particular, the guidewires described herein can have a longer conductive length, such that when the guidewires are extended into the blood pool beyond the septum, the longer conductive length of the guidewires spreads out the current density along the length of the guidewire. This then reduces the current density at any point along the guidewire, thereby reducing the risk of injury to the myocardium, should the guidewire come into contact with the heart wall.

For example, a guidewire can include first distally electrically conductive portion that has a length of about 1.0 mm, a first insulating region that has a length of about 3 mm to 1 cm, and a second conductive region that has a length of about 12.0 cm. The first conductive portion and the second conductive portion can include metallic structures including coils (e.g., stainless steel coils, tungsten coils, etc.) while the insulating region can include a polymeric jacketing materials, such as polyolefin, polyethylene terephthalate, and/or the like. When the distal tip of the guidewire extends a distance of less than about 5 mm beyond the dilator, the tip of the guidewire can be configured to have a current density of between about 10 A/cm² to about 250 A/cm², including all subranges and values therebetween. In embodiments, the current density can be greater than 10 A/cm² when the tip is extended a first distance of less than 1 mm beyond the dilator. Further, the tip of the guidewire can be configured to have a current density of less than 60 A/cm² when the distal end of the guidewire extends less than about 5 mm beyond the dilator. For example, the current density can be about 60 A/cm² when the distal tip is extended less than or equal to about 0.5 mm of the sheath. Further, the tip of the guidewire can be configured to have a current density of less than about 50 A/cm², or less than about 40 A/cm², or less than about 30 A/cm², or less than about 25 A/cm², or less than about 20 A/cm², or less than about 15 A/cm², or less than about 10 A/cm², or less than about 5 A/cm², when the tip extends beyond about 5 mm beyond the dilator. For example, when the tip extends a second distance greater than 7 mm beyond the dilator, the tip can have a current density of less than about 30 A/cm².

### Energy Delivery

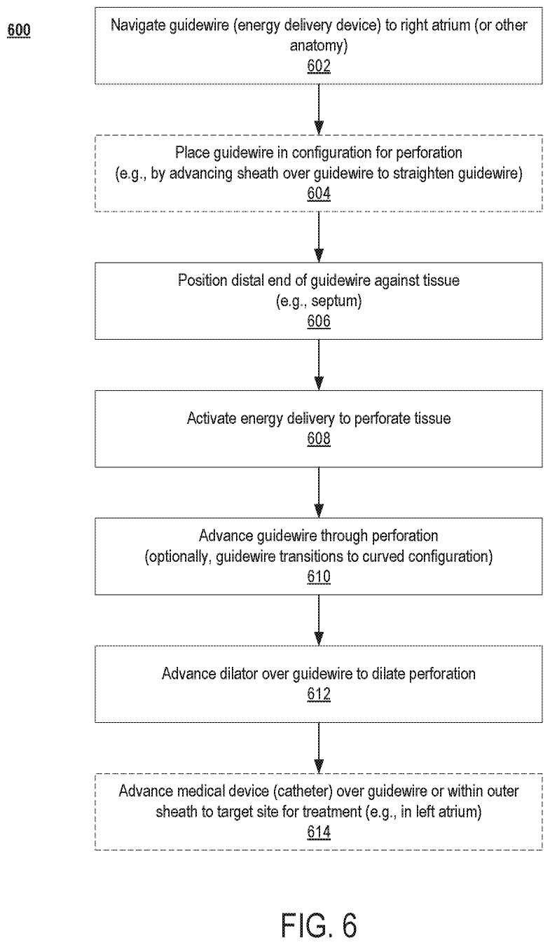

FIG. 6 is flow chart of a method 600 of using the systems and devices described herein, according to embodiments. The method 600 includes navigating a guidewire (e.g., functionally and/or structurally similar to the energy delivery element 124 of FIG. 1A or other guidewires described herein) through vasculature to a right atrium (or other anatomy) of a patient at 602, optionally placing the guidewire in configuration for perforation (e.g., by advancing a sheath over guidewire to straighten the guidewire) at 604, positioning a distal end of the guidewire against tissue (e.g., a septum) at 1206, activating energy delivery to perforate the tissue at 608, advancing the guidewire through the perforation at 610, advancing the dilator over the guidewire to dilate the perforation at 612, and optionally advancing a medical device (e.g., catheter) over the guidewire to a target site for treatment at 614.

At 602, the guidewire is navigated to a target location in a patient's body. The target location may be the right atrium of the heart or other patient anatomy (e.g., in the heart, vasculature, or other anatomy). In some embodiments, the guidewire is inserted into the femoral vein via a puncture created by a standard needle puncture technique. The guidewire is navigated or advanced through the vasculature using fluoroscopic and/or ultrasonic imaging. The guidewire can be navigated through the vasculature and positioned above the right atrial chamber in the superior vena cava (SVC).

Before or after navigating the guidewire to the right atrium (or other anatomy), the guidewire may be connected to a generator, e.g., via an electrosurgical interface (e.g., electrosurgical interface 122, 222, etc.). To connect the guidewire to receive energy from the generator, an intervascular sheath and/or dilator (e.g., sheath 126, dilator 326, dilator 426 etc.) can be prepared by attaching the electrosurgical interface to the lumen of the sheath via standard Luer connections. In some embodiments, the fluid lumen is flushed with a sterile 0.9% sodium chloride saline solution such that air in the lumen is removed. The sheath is then loaded on the proximal end of the guidewire and advanced into the patient's vasculature such that the distal tip of the dilator is positioned in the right atrium.

In some embodiments, as described above, the guidewire can have a J-shape or other atraumatic distal end shape. In such embodiments, it may be necessary to first place the guidewire in a specific configuration before the guidewire can be used to perforate through tissue. As such, at 604, the guidewire optionally is placed in a configuration for perforation (e.g., a straightened or substantially straightened configuration). In some embodiments, placing the guidewire in the configuration for perforation includes advancing the sheath as described above over the distal curved portion of the guidewire to straighten the guidewire. In some embodiments, placing the guidewire in the configuration for perforation includes retracting the guidewire into the sheath such that the guidewire straightens. When positioned for perforation, the guidewire tip can be exposed about 1 to about 2 mm from the distal end of the dilator. Alternatively, in some embodiments, the guidewire may not have a J-shape construction. In such embodiments, it may not be necessary to move the guidewire relative to the sheath to straighten the guidewire or place it into a configuration for perforation. As such, 604 can be omitted.

At 606, the distal end (e.g., tip) of the guidewire is positioned against tissue. In some embodiments, the tissue is a portion of the interatrial septum of the heart. In some embodiments, the sheath may be a steerable sheath and/or dilator and therefore can be utilized to position or direct the guidewire toward the tissue, e.g., by actuating a steering mechanism (e.g., pull wires) on the sheath to deflect the distal portion of the sheath. In some embodiments, a pull-down technique is performed, in which the distal end of the dilator is directed toward the fossa ovalis (FO), and tenting of the tissue (e.g., septum) is facilitated via fluoroscopic and/or ultrasound imaging (e.g., as shown in FIG. 3B). In some embodiments, repositioning of the dilator may be desired by the operator after performing the tenting. To reposition, the guidewire may be re-advanced into the SVC and the pull-down technique may be repeated.

At 608, energy delivery is activated to perforate the tissue. The perforation is formed by the tip of the guidewire delivering energy to the tissue and forming the perforation. In some embodiments, the electrosurgical energy is applied by actuating a button or other actuator connected to the electrosurgical interface. In some embodiments, the perforation is formed by applying energy and applying a slight pressure on the tissue with the guidewire. Energy delivery is stopped once a puncture is formed within the tissue. In some embodiments, the formation of the puncture may be confirmed via imaging and/or tactile feel.

At 610, the guidewire is advanced through the perforation formed in 608. In some embodiments, the guidewire transitions to back to its curved configuration (e.g., as shown in FIG. 3C). In some embodiments, the guidewire is advanced through the FO and positioned in the left atria or the pulmonary veins. At 612, the sheath implemented with a dilator can be advanced over the guidewire to dilate the perforation. In some embodiments, the dilator is advanced through the FO and positioned in the left atrium.

In some embodiments, the dilator and the electrosurgical interface can be retracted and removed, and additional devices (e.g., catheter and/or sheaths) can be advanced over the guidewire and into the left atrium (or other target anatomy), at 614.

In some embodiments, an outer sheath may be positioned around the dilator and/or guidewire and advanced into the left atrium, e.g., at the same time as the dilator or shortly thereafter. This outer sheath may be positioned over the dilator and/or guidewire after the perforation has been formed and dilated and the electrosurgical interface has been removed from the proximal end of the dilator. Alternatively, this outer sheath may have been positioned over the dilator from the beginning of the procedure and used together with the dilator at 604, 606, and 612. In such embodiments, the outer sheath can be used to provide an access passage or channel into the left atrium. The dilator and the guidewire are removed from the outer sheath, while the sheath lumen remains in the left atrium. At 614, a medical device (e.g., a catheter, etc.) is advanced through the sheath lumen and to a target site for treatment.

In some embodiments, a therapeutic ablation catheter is advanced into the left atrium so that a pulmonary vein isolation may be performed. Once a procedure is performed, the medical device is removed.

Referring generally to FIGS. 7 and 8, it may be beneficial to alter the energy delivered from a generator (e.g., functionally and/or structurally similar to the generator 110 of FIG. 1A) to the guidewire (e.g., functionally and/or structurally similar to the energy delivery element 124 of FIG. 1A). In some embodiments, it may be desirable to alter the energy being delivered to the guidewire tip based on measured properties such as, for example, temperature. This can help avoid buildup of heat at a distal tip of the guidewire, thereby preventing undesirable formation of microbubbles. In some embodiments, it may be desired for the guidewire to deliver a varied amount of energy to the tissue. For example, when the guidewire's distal tip is in-line or slightly past the tip of the sheath (e.g., about 1 mm), the guidewire has a small effective surface area, which can cause a high potential current density and thus heating near the guidewire tip. As the guidewire extends out of the sheath, the exposed surface area of the guidewire increases, thereby decreasing current density. To control the potential changes in current density, it may be beneficial to alter the energy delivered to the guidewire to prevent inadvertent damage to the tissue by the guidewire.

FIG. 7 depicts a flowchart of a method 700 of varying an RF output being delivered to a guidewire, according to embodiments. The method 700 includes modifying the output based on at least one characteristic associated with the guidewire. In some embodiments, the guidewire can include at least one sensor to measure the at least one characteristic (e.g., a state of the guidewire, temperature). In some embodiments, the sensor can be configured to capture data indicative of the at least one characteristic and send that data to the generator, which in response to receiving the data, may adjust the energy being delivered to the guidewire.

At 702, the method 700 includes delivering, via an RF generator, RF output to a guidewire. In some embodiments, the amount of RF output delivered to the guidewire can be predetermined. In some embodiments, the amount of RF output delivered to the guidewire can be determined based on a predetermined parameter (e.g., patient parameter, device parameter, etc.). At 704, the method 700 includes detecting at least one characteristic associated with the guidewire. In some implementations, the at least one characteristic can be an RF current, an RF output, temperature, current density, pressure, current, voltage, and/or the like. In some implementations, the at least one characteristic associated with the guidewire can be a state of the guidewire. For example, the state can include: guidewire inside a sheath; guidewire starting to advance toward the distal end of a sheath or dilator; guidewire engaging tissue, and/or the like. As another example, the at least one characteristic can include current density at/around the tip of the guidewire.

At 706, the method 700 includes modifying the RF output based on the at least one characteristic. Modifying the RF output can include modifying an output power, peak-to-peak voltage, duty-cycle, and/or the like. For example, if the temperature at the tip of the guidewire is determined to be greater than a predetermined threshold, the RF output can be modified to deliver less power to the guidewire. As another example, if a pressure sensor detects that the septum of a heart has been punctured, the RF output can be decreased to decrease the likelihood of accidental damage. As another example, the RF output can be modified based on the current density at/around the tip of the guidewire and based on the location of the guidewire tip. For example, if the guidewire tip is in contact with the heart wall and the current density is greater than desired (e.g., such that it may form a lesion, the RF output can be decreased. After the RF output is modified, the method 700 includes delivering the modified RF output to the guidewire at 708. After the RF output is delivered to the guidewire, the method 700 may return to 704 to again detect at least one characteristic associated with the guidewire. Repeating steps 704-708 allows the system to dynamically change based on the detected at least one characteristic to decrease the likelihood of damage tissue.

FIG. 8 depicts a flowchart of a method 800 of delivering an RF output to a guidewire, according to embodiments. The method 800 includes altering the output to the guidewire based on an output schedule. This allows a predetermined amount of energy to be delivered during different stages of a procedure. In some embodiments, the output schedule is predetermined. In some embodiments, the treatment schedule can be modified based on the patient, device, and/or the like. In some embodiments, the treatment schedule can be defined by a user. At 802, the method 800 includes delivering, via an RF generator, RF output to a guidewire at first power parameters for a first period of time. At 804, the method 800 includes delivering, via the RF generator, RF output to the guidewire at a second power parameters for a second period of time. At 806, the method 800 optionally includes delivering, via the RF generator, RF output to the guidewire at a third power parameters for a third period of time. In some embodiments, the method 800 can include additional steps for delivering RF output to the guidewire at additional power parameters for additional periods of time. The power parameters can correspond to duty-cycles. The periods of time can correspond to durations associated with the operation of the guidewire. For example, the first power parameters can include a 10% duty cycle, the second power parameters can include a 50% duty cycle while the first period of time is 25% of the duration of treatment and the second period of time is 75% of the duration of treatment.

Additionally or alternatively, in some embodiments, a generator (e.g., generator 110 as described herein) can detect when the current density at the distal end of the guidewire decreases (e.g., as the conductive region of the surface area increases due to cutting through tissue and advancing into blood, e.g., into the left atrium) and can adapt to deliver more current up to a predefined or user-programmed limit. When the wire has lower surface area exposed, less current may be needed, but as the wire extends into the tissue, the increasing surface area may need more current to maintain a current density effective for perforating through the tissue. When the wire is fully deployed (e.g., 1 cm from the dilator or other insulating shaft tip), the current density is too low to cause inadvertent tissue damage, even with the higher RF output.

FIG. 9 is as flow chart depicting a method 900 of adjusting energy delivered to an electrosurgical device (e.g., structurally and/or functionally similar to the electrosurgical device 120 of FIG. 1 and/or the electrosurgical device 220 of FIG. 2) by a generator (e.g., structurally and/or functionally similar to generator 110 of FIG. 1 and/or the generator 210 of FIG. 2), according to embodiments. The method 900 can be an example of an automatic energy shut off algorithm. The method 900 includes measuring voltage (V) and current (I) over a period of time at 902, optionally determining V_{RMS} and I_{RMS} at 904, optionally determining complex V and complex I at 906, optionally determining phase using complex V and complex I at 908, determining impedance magnitude (Z_{mag}) at 910, optionally determining apparent power (P_{Apparent}) at 912, optionally determining real power (Pᵣₑₐₗ) at 914, optionally determining a power factor (PF) at 916, optionally determining a complex impedance (Zᵢₘ and Zᵣₑ) at 918, optionally determining resistance associated with an energy delivery element (e.g., functionally and/or structurally similar to the energy delivery element 124 of FIG. 1, the energy delivery element 224 of FIG. 2, the energy delivery element 324 of FIGS. 3A-3C, and/or the energy delivery element 424 of FIG. 4A-4C) tip (Rₚ) at 920, optionally determining capacitance associated with a sheath (Cₚ) (e.g., functionally and/or structurally similar to the sheath 126 of FIG. 1, the dilator 326 of FIGS. 3A-3C, and/or the dilator 426 of FIGS. 4A-4C) at 922, determining a state of the electrosurgical device at 924, and adjusting energy delivered to the electrosurgical device based on the state at 926. Similarly the method 700 of FIG. 7 and the method 800 of FIG. 8, the method 900 includes altering the operation of the electrosurgical device to reduce the likelihood of undesired damage to the tissue, decrease or prevent coagulation, and/or provide desired conditions for puncturing through the tissue. In some embodiments, 904 through 924 may be done serially, in parallel, or in combinations thereof. The method 700, method 800, and/or the method 900 may be operating simultaneously, individually, or any combination. In some embodiments, the method 700, method 800, and/or the method 900 can be executed by the generator automatically during operation. Generally, the method 900 includes determining a number of electrical parameters, some of which can be optional. In some embodiments, the method 900 includes determining electrical parameters that are used to determine states of the electrosurgical device and/or for adjusting energy.

At 902, the voltage (V) and the current (I) is measured over a period of time. In some embodiments, the period of time can be predetermined. In some embodiments, the period of time can be associated with a procedure. In some embodiments, the voltage and/or the current can be measured continuously throughout a procedure. In some embodiments, the voltage signal and the current signal may be synced so that the signals and/or other parameters can be compared. In some embodiments, the voltage is measured by a voltmeter included in or coupled to the circuit formed by the generator and the electrosurgical device. In some embodiments, the current is measured by an ammeter included in or coupled to the circuit. In some embodiments, additional parameters associated with the circuit can be measured, such as the frequency of the generator, and/or the like. In some embodiments, the measured voltage and current can be associated with a series representation of the circuit as seen in FIG. 14A. At 904, the RMS values of the voltage and the current can be determined to define an RMS voltage (V_{RMS}) and an RMS current (I_{RMS}).

At 906, the method 900 includes determining complex V (V_{complex}), as seen in Equation 1 and complex I (I_{complex}), as seen in Equation 2, where j=\sqrt{−1}, a represents the real component of the voltage, jb represents the imaginary component of the voltage, c represents the real component of the current, and jd represents the imaginary component of the current.

$${V}_{complex} = a + jb \qquad ( 1 )$$

$${I}_{complex} = c + jd \qquad ( 2 )$$

The complex V and complex I can be determined based on the generator using Goertzel's algorithm, a Fourier transform (e.g., fast Fourier transform, etc.), and or other method for determining the complex V and the complex I. In some embodiments, the magnitude of the complex V and the magnitude of the complex I can be determined. In some embodiments, the magnitude of the complex V and the magnitude of the complex I can be substantially equal to the RMS voltage and/or the RMS current. In some embodiments, determining the complex V and the complex I may be optional.

At 908, the complex V and the complex I can be used to determine the phase angle (φ) using Equations 3-5. Equation 3 is the dot product of the complex V and the complex I, Equations 4 is the determinant of the complex V and the complex I, and Equation 5 is the phase angle determination based on the outputs of the dot product and determinant. In some embodiments, the value of the phase angle can be associated with the guidewire engaging the tissue. In some embodiments, determining the phase angle is optional.

$$x = a * c + b * d \qquad ( 3 )$$

$$y = a * d - b * c \qquad ( 4 )$$

$$ϕ = atan 2 ( y , x ) \qquad ( 5 )$$

At 910, the impedance magnitude (Z_{mag}) is determined. As seen below in Equation 6, the impedance magnitude can be determined based on the RMS voltage and the RMS current. In some embodiments, the impedance magnitude may be determined based on the magnitude of the complex voltage and the complex current.

$${Z}_{mag} = \frac{{V}_{RMS}}{{I}_{RMS}} \qquad ( 6 )$$

In some embodiments, the impedance magnitude can be associated with the impedance of a series representation of the circuit as seen in FIG. 14A. In some embodiments, the impedance magnitude can be associated with the guidewire engaging the tissue. For example, the impedance magnitude may decrease from the guidewire being in the sheath, to extended beyond the sheath, to engaging the tissue, to the blood pool after puncturing the tissue. In some embodiments, determining the impedance magnitude is optional.

At 912, the apparent power (P_{Apparent}) can be determined based on the RMS voltage and the RMS current as in Equation 7. In some embodiments, determining the apparent power can be based on the magnitude of the complex voltage and the magnitude of the complex current.

$${P}_{Apparent} = {V}_{RMS} * {I}_{RMS} \qquad ( 7 )$$

In some embodiments, the apparent power can correspond to the total power generated by the generator during operation. In some embodiments, the apparent power can be set on the generator by a user. In some embodiments, the apparent power can be constant during a procedure. In some embodiments, determining the apparent power is optional.

At 914, the real power (Pᵣₑₐₗ) can be determined based on the apparent power and the phase angle as seen in Equation 8.

$${P}_{real} = {P}_{Apparent} * cos ( ϕ ) \qquad ( 8 )$$

In some embodiments, the real power can be associated with the power delivered by the energy delivery element. For example, the real power can be associated with the energy delivered to the tissue, the blood pool, and/or the like. In some embodiments, the real power can be associated with the power delivered across the wire, tissue, blood pool, and/or the like as shown in the parallel representation of FIG. 14B. The real power can be associated with whether the guidewire has engaged the tissue as an increased real power can indicate that the guidewire has engaged the tissue or the blood pool. For example, as the phase angle approaches 90 degrees, minimal power can be dissipated by the tissue and the majority of the current may be flowing through the sheath. As the guidewire reaches the tissue, the real power can increase, and thus the phase angle decreases. In some embodiments, determining the real power is optional.

At 916, the power factor can be determined based on the phase angle, as seen in Equation 9. The power factor is associated with the ratio between the real power and the apparent power. In some embodiments, the determining the power factor is optional.

$$P F = cos ( ϕ ) \qquad ( 9 )$$

At 918, the complex impedance is determined including the real component of the complex impedance (Zᵣₑ) and the imaginary component of the complex impedance (Zᵢₘ) based on the impedance magnitude and the phase angle as seen in Equations 10-11. In some embodiments, determining the complex impedance can be optional.

$${Z}_{re} = {Z}_{mag} * cos ( ϕ ) \qquad ( 10 )$$

$${Z}_{im} = {Z}_{mag} * sin ( ϕ ) \qquad ( 11 )$$

At 920, the resistance associated with the electrosurgical device tip (Rₚ) is determined based on the complex impedance as seen in Equation 12.

$${R}_{p} = \frac{{Z}_{re}^{2} + {Z}_{im}^{2}}{Zre} \qquad ( 12 )$$

In some embodiments, the resistance corresponds to the resistance across the wire and the tissue as in the parallel representation of the circuit as seen in FIG. 14B. In some embodiments, the resistance can indicate if the tip of the electrosurgical device is in the sheath, at the tissue, and/or in the blood pool. In some embodiments, determining the resistance can be optional.

At 922, the capacitance associated with the sheath (Cₚ) can be determined using Equations 13-14. The determination of capacitance can be used for error checking, e.g., if the capacitance is outside an expected value (which in some embodiments may be, for example, 150 to 250 pf), or to enable identification of the sheath type and/or other characteristics of the system or setup. Equation 13 determines the reactance and Equation 14 determines the capacitance based on the reactance. In some embodiments, the reactance represents the capacitive reactance of the wire in the insulating sheath to the return electrode. In some embodiments, the capacitance may correspond to a capacitance across the sheath as in the parallel representation of the circuit as seen in FIG. 14B. In some embodiments, the capacitance across the sheath may be constant. In some embodiments, determining the capacitance may be optional.

$${X}_{p} = \frac{{Z}_{re}^{2} + {Z}_{im}^{2}}{{Z}_{im}} \qquad ( 13 )$$

$${C}_{p} = \frac{- 1}{2 * π * f * {X}_{p}} \qquad ( 14 )$$

At 924, a state of the electrosurgical device can be determined. The state can correspond to a position of one or more component of the electrosurgical device relative to the tissue, left atrium, blood pool, heart wall, and/or the like. In some embodiments, a plurality of states of a distal conductive region (e.g., long electrode) of the electrosurgical device can be determined over a period of time. The states of the electrosurgical device are further described in reference to FIG. 10. At 924, the state of the electrosurgical device can be determined based on one or more of the parameters determined in method 900. For example, the state of the electrosurgical device can be determined based on one or more of the voltage, current, RMS voltage, RMS current, impedance magnitude, complex impedance, phase, real power, resistance, capacitance, and/or the like. In some embodiments, the state of the electrosurgical device can be determined based on the value, rate of change, and/or the like of the parameters. In some embodiments, determining the state can be based on a past state determination. For example, if a past state indicates that the electrosurgical device has puncture the tissue and is in the left atrium, an increase in resistance can indicate that the electrosurgical device has contacted the heart wall. Alternatively, if the past state indicates that the tissue has not yet been punctured, an increase in resistance can indicate that the guidewire has been pulled away from the tissue. In some embodiments, determining the states can be based on threshold associated with the one or more parameters. In some embodiments, determining the state can be based on multiple parameters to decrease the likelihood of a false determination.

For example, the RMS voltage and RMS current can indicate whether the guidewire is in the sheath, engaging tissue, or in the blood pool. For example, a high voltage can indicate that the guidewire is in the sheath, a lower voltage can indicate that the guidewire has engaged the tissue, and an even lower voltage can indicate that the guidewire has entered the blood pool. In some embodiments, an increase in voltage can indicate that the guidewire is pulled back into the sheath after engaging the tissue. In some embodiment, the RMS current indications of the state can be inverse to the RMS voltage. For example, a decrease in current can indicate a similar state change to an increase in voltage. In some embodiments, additional states can be determined based on the RMS voltage and the RMS current.

As another example, the impedance magnitude can be used to determine the location of the guidewire in the sheath, engaging tissue, or in the blood pool. For example, a high impedance magnitude can indicate that the guidewire is in the sheath. In some embodiments, the impedance can decrease as the guidewire is pushed toward the distal tip of the sheath, thus indicating the depth of the guidewire in the sheath. A drop in the impedance can indicate that the guidewire has been extended beyond the distal tip of the sheath and has engaged the tissue. Another drop in the impedance can indicate that the guidewire is in the blood pool. In some embodiments, additional states can be determined based on the impedance magnitude.

As another example, the real power can be used to determine the state of the guidewire in the sheath, engaging tissue, or in the blood pool. As the real power indicates the power delivered across the tip of the guidewire and the tissue/blood pool, the real power can indicate, when the real power increases from a low amount, that the guidewire has engaged the tissue as the tissue has a lower resistance than the sheath. When the real power again increases, it can be determined that the guidewire is in the blood pool. In some embodiments, the resistance can have an inverse relationship to the real power, where high power indication corresponds to low resistance values.

At 926, the energy delivered to the electrosurgical device is adjusted based on the state. In some embodiments, the state can indicate that no change in the energy delivery is desired. In some embodiments, adjusting can include stopping the energy delivery. In some embodiments, adjusting the energy delivery can be based on the state corresponding to a successful operation. For example, a successful operation can include the guidewire successfully puncturing through the tissue. In some embodiments, adjusting the energy delivery can be based on the state corresponding to a potentially dangerous state which can include failing to puncture or after a successful puncture. The energy delivery can be stopped during the potentially dangerous situations to decrease the likelihood of undesired damage to the heart. In some embodiments, adjusting the energy delivery can be based on one or more parameters and one or more thresholds regardless of the state determination. For example, if the current and/or voltage are above a predetermined threshold, the energy delivery can be stopped to reduce the likelihood of undesired tissue damage.

FIG. 10 depicts different states of a distal end of an electrosurgical device (e.g., functionally and/or structurally similar to the electrosurgical device 120) used for perforating tissue, according to embodiments. The electrosurgical device includes a guidewire (e.g., functionally and/or structurally similar to the guidewire 124, guidewire 224, the guidewire 324, and/or the guidewire 424) at least partially disposed within a sheath (e.g., functionally and/or structurally similar to the sheath 126, the dilator 326, and/or the dilator 426). The states depicted are organized in three groups: states associated with the right atrium (1, 2, 3, 4, 5, 9 and 10), states associated with the tissue (6, 7, and 8), and states associated with the left atrium (11, 12, 13, 14, 15, and 16). In some embodiments, additional states may also exist when an electrosurgical device engages tissue. In some embodiments, the states can be associated with values, rates of changes, and/or the like of various electrical parameters when the generator is operating.

Generally referring to the states associated with the right atrium, the sheath and the guidewire are within the right atrium and are either engaging the tissue or adjacent to the tissue. State 1 depicts the guidewire extended beyond the distal end of the sheath, but not engaging the tissue. In some embodiments, state 1 can be associated with a substantially low resistance value, a relatively low voltage value, a relatively high real power, a relatively low impedance, and a relatively high current value as the guidewire is exposed to the blood pool in the right atrium but not engaged the tissue.

State 2 depicts the guidewire stationary within the sheath, while the sheath is not engaging the tissue. In some embodiments, state 2 can be associated with a relatively high voltage value, a relatively low current value, a relatively high resistance value, a relatively high impedance magnitude, and a relatively low real power as the guidewire wire is disposed within the sheath and not exposed to the blood pool or the tissue.

State 3 depicts the sheath contacting the tissue as the guidewire is advanced toward the tissue. In some embodiments, state 3 can be associated with a relatively high voltage value, a relatively low current value, a relatively high resistance, a relatively high but decreasing impedance magnitude, and a relatively low real power as the guidewire is disposed within the sheath and not exposed to the blood pool or the tissue.

State 4 depicts the sheath contacting the tissue as well as the guidewire. In some embodiments, state 4 can be associated with an intermediate voltage value, an intermediate current value, an intermediate resistance, an intermediate impedance magnitude, and an intermediate real power as the guidewire is disposed within the sheath and engaging the tissue and delivering energy to the tissue.

State 5 depicts the guidewire engaging the tissue and the sheath being pulled back from the tissue. In some embodiments, state 5 can be associated with an intermediate voltage value, an intermediate current value, an intermediate but decreasing resistance, an intermediate but decreasing impedance magnitude, and an intermediate real power as the guidewire is engaging the tissue and is exposed to an increasing portion of the blood pool.

State 6 depicts the sheath contacting the tissue as the guidewire has advanced partially through the tissue. In some embodiments, state 6 can be associated with an intermediate voltage value, an intermediate current value, an intermediate resistance, an intermediate impedance magnitude, and an intermediate real power as the guidewire is disposed within the tissue and the guidewire is shielding the rest of the guidewire from the blood pool.

State 7 depicts the sheath contacting the tissue as the guidewire is advancing through the tissue. In some embodiments, state 7 can be associated with an intermediate voltage value, an intermediate current value, an intermediate resistance, an intermediate impedance magnitude, and an intermediate real power as the guidewire is disposed within the tissue and the guidewire is shielding the rest of the guidewire from the blood pool.

State 8 depicts the sheath contacting the tissue as the guidewire has nearly punctured through the tissue. In some embodiments, state 8 can be associated with an intermediate voltage value, an intermediate current value, an intermediate resistance, an intermediate impedance magnitude, and an intermediate real power as the guidewire is disposed within the tissue and the guidewire is shielding the rest of the guidewire from the blood pool.

State 9 depicts the sheath contacting the tissue at an angle as the guidewire fails to puncture through and deflects off the tissue. In some embodiments, state 9 can be associated with an intermediate voltage value, an intermediate current value, an intermediate resistance, an intermediate impedance magnitude, and an intermediate real power as the guidewire is engaging the tissue and is exposed to a portion of the blood pool.

State 10 depicts the sheath contacting the tissue at an angle as the guidewire fails to puncture through and deflects off the tissue and continues into the blood pool. In some embodiments, state 10 can be associated with an intermediate voltage value, an intermediate current value, a relatively low resistance, a relatively low impedance magnitude, and an intermediate real power as the guidewire is engaging the tissue and is exposed to a portion of the blood pool. In some embodiments, it may be desirable to stop energy delivery to the guidewire to avoid undesirable tissue damage.

State 14 depicts the guidewire extended beyond the distal end of the sheath and being further retracted, but not engaging the tissue. In some embodiments, state 14 can be associated with a substantially low resistance value, a relatively low voltage value, a relatively high real power, a relatively low impedance, and a relatively high current value as the guidewire is exposed to the blood pool in the right atrium but not engaged the tissue.

Generally referring to the states associated with the tissue, the sheath and the guidewire have punctured the tissue but has not fully extended in the left atrium. In some embodiments, it may be desirable to stop delivering energy to the guidewire after the guidewire has punctured through the tissue. State 11 depicts the sheath against the tissue and the guidewire punctured through the tissue and in the left atrium. In some embodiments, state 11 can be associated with a relatively low voltage value, a relatively high current value, a relatively low resistance, a relatively low impedance magnitude, and a relatively high real power as the guidewire is exposed to a portion of the blood pool.

State 12 depicts the sheath against the tissue and the guidewire punctured through the tissue and advancing in the left atrium. In some embodiments, state 12 can be associated with a relatively low voltage value, a relatively high current value, a relatively and decreasing low resistance, a relatively low and decreasing impedance magnitude, and a relatively high real power as the guidewire is exposed to an increasing portion of the blood pool.

State 13 depicts the sheath being pulled back from the tissue and the guidewire punctured through the tissue and in the left atrium. In some embodiments, state 13 can be associated with a relatively low voltage value, a relatively high current value, a relatively and decreasing low resistance, a relatively low and decreasing impedance magnitude, and a relatively high real power as the guidewire is exposed to an increasing portion of the blood pool.

Generally referring to the states associated with the left atrium, the guidewire is fully deployed in the left atrium after puncturing through the tissue. State 15 depicts the guidewire contacting a portion of the heart wall not associated with the septum, which can be hazardous and is thus undesirable. In some embodiments, state 15 can be associated with an intermediate voltage value, an intermediate current value, an intermediate resistance, an intermediate impedance magnitude, and an intermediate real power as the guidewire is engaging the tissue of the heart wall not associated with the septum but exposed to the blood pool.

State 16 depicts the sheath against the tissue and the guidewire punctured through the tissue and in the left atrium. In some embodiments, state 16 can be associated with a relatively low voltage value, a relatively high current value, a relatively low resistance, a relatively low impedance magnitude, and a relatively high real power as the guidewire is exposed to the blood pool. In some embodiments, it may be desirable for energy to be shut off at or before states 15 and 16 to prevent inadvertent damage to the tissue such as when the guidewire contacting the heart wall in state 15 or to reduce the formation of coagulum in state 16.

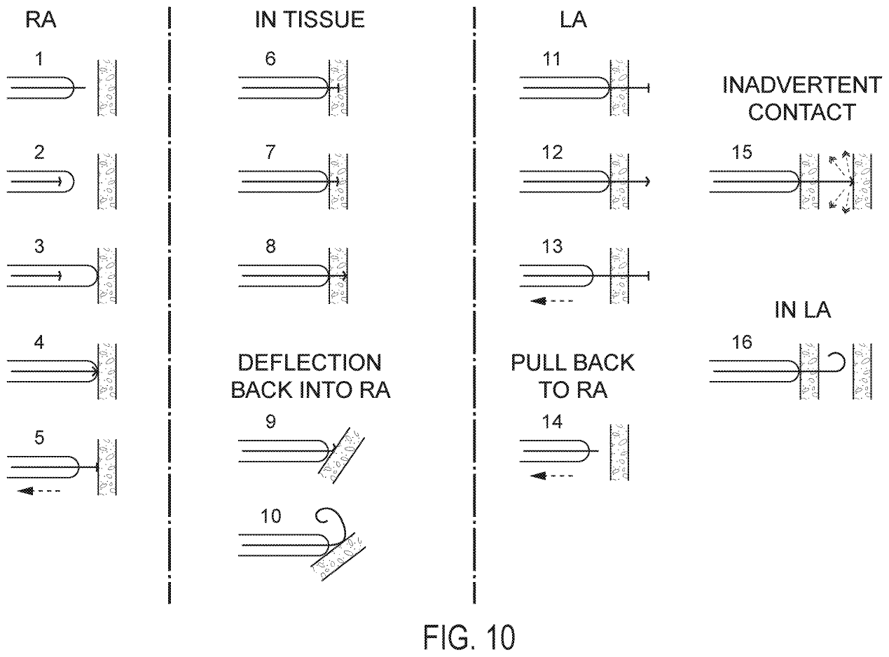

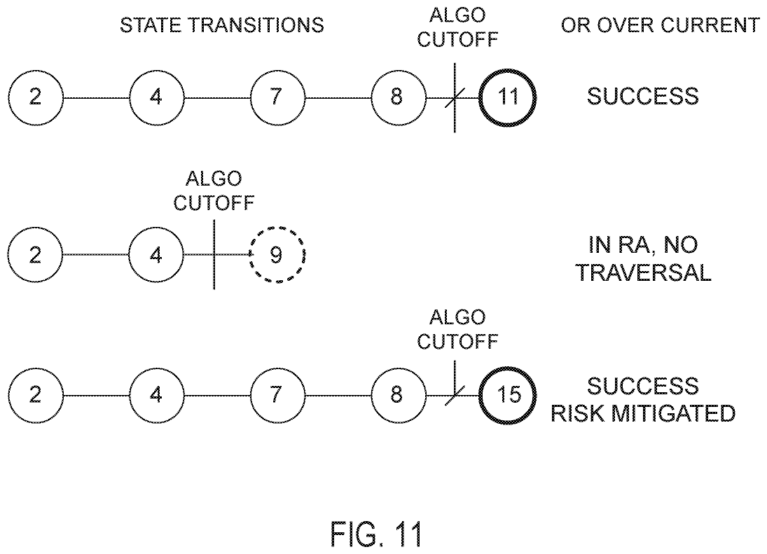

FIG. 11 depicts various embodiments of state (e.g., the states of FIG. 10) transitions, according to embodiments. The state transitions describe different tissue puncturing situations and applying an energy delivery cutoff as described in reference to FIG. 9 based on the state. A first state transition can include beginning at state 2 with the guidewire disposed fully in the sheath and the sheath not engaging the tissue. Then, proceeding to state 4, wherein the sheath and the guidewire engage the tissue. At state 4, energy can be delivered, via the guidewire to the tissue to begin puncturing through the tissue and proceeding to state 7, during which the guidewire advances through the tissue until reaching state 8 where the tissue is punctured. After the tissue is punctured, the generator can determine an energy delivery cutoff prior to the guidewire being advanced to state 11. The first state transition describes a successful puncturing of the tissue without any undesirable damage.

A second state transition can include beginning at state 2 with the guidewire disposed fully in the sheath and the sheath not engaging the tissue. Then, proceeding to state 4, wherein the sheath and the guidewire engage the tissue. At state 4, energy can be delivered, via the guidewire to the tissue to begin puncturing through the tissue. However, the tissue my be angled relative to the tip of the guidewire and the guidewire may fail to puncture and the generator can determine an energy delivery cut off prior to the guidewire entering the blood pool in the RA in state 9. The energy cutoff in the second state transition can decrease the likelihood that a failed puncture causes undesired damage in the right atrium and reduces the formation of coagulum.

A third state transition, similar to the first state transition, can include beginning at state 2 with the guidewire disposed fully in the sheath and the sheath not engaging the tissue. Then, proceeding to state 4, wherein the sheath and the guidewire engage the tissue. At state 4, energy can be delivered, via the guidewire to the tissue to begin puncturing through the tissue and proceeding to state 7, during which the guidewire advances through the tissue until reaching state 8 where the tissue is punctured. After the tissue is punctured, the generator can determine an energy delivery cutoff prior to the guidewire being advanced into the heart wall at state 15, thus avoiding undesired damage to the heart wall and mitigating the risk. In some embodiments, additional state transitions can be used during a procedure to successfully puncture the tissue and reduce the risk of undesired damage to the heart.

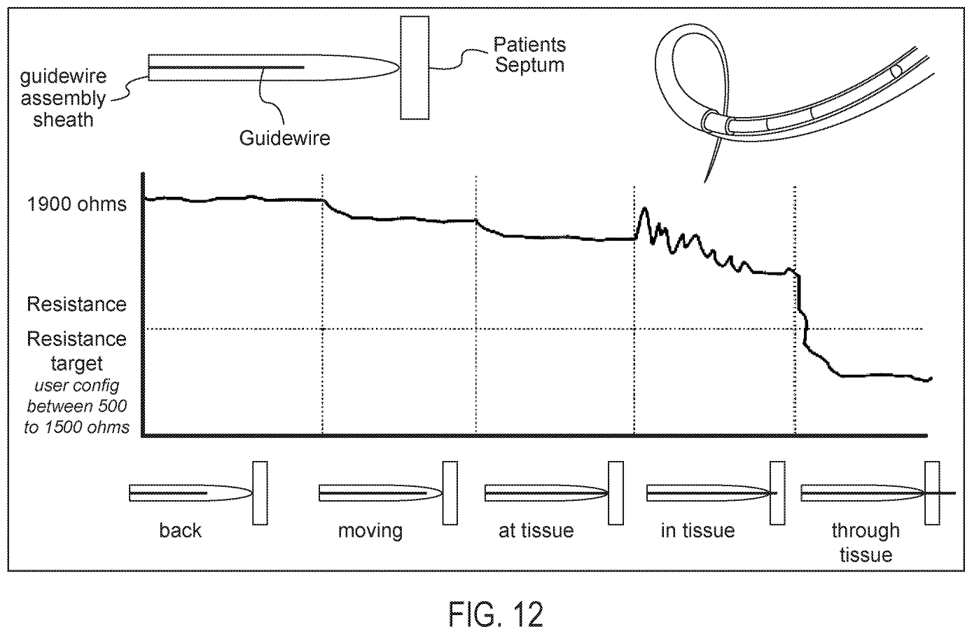

FIG. 12 depicts a graph of resistance as measured by a generator (e.g., structurally and/or functionally similar to the generator 110, the generator 120, and/or any of the generators described herein) as an electrosurgical device (e.g., functionally and/or structurally similar to the electrosurgical device 120, the electrosurgical device 220, and/or any of the electrosurgical devices described herein) engages tissue (e.g., a septum of a patient), according to an embodiment. As seen in FIG. 12, the electrosurgical device includes a guidewire (e.g., functionally and/or structurally similar to the energy delivery element 124, the energy delivery element 224, the guidewire 324, the guidewire 424, and/or the guidewire 1724) disposed, at least partially, within a sheath (e.g., functionally and/or structurally similar to the sheath 126, the dilator 326, and/or any sheath described herein).

The graph shown in FIG. 12 is associated with five states of the guidewire as the sheath is engaging the tissue. A first state includes the guidewire in shielded in the back of the sheath. During the first state, the resistance is approximately 1900 ohms. A second state includes the guidewire moving toward the tissue. During the second state, the resistance decreases from the first state. A third state includes the guidewire at the tissue. During third state, the resistance further decreases from the second state. A fourth state includes the guidewire puncturing through the tissue. During the fourth state, the resistance first increases above the resistance of the third state then decreases to lower than the fourth state as the guidewire punctures through the tissue. A fifth state includes the guidewire having punctured through the tissue. During the fifth state, the resistance drops under a resistance target (e.g., between about 100 to 1500 Ohms) as the guidewire enters the blood pool. In some embodiments, the energy delivery can be stopped based on the resistance being measured as below at or below the resistance target.

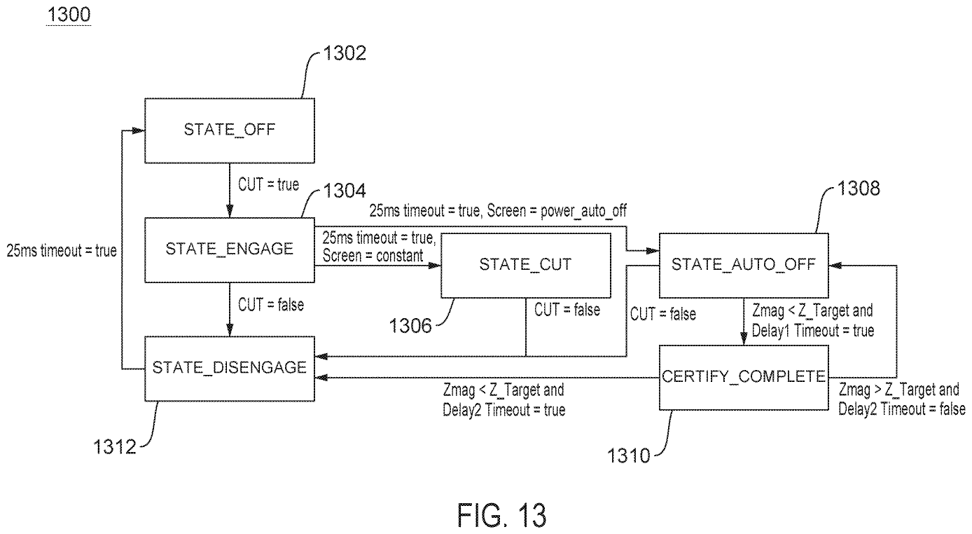

FIG. 13 depicts a flow diagram of a method 1300 for operating a generator (e.g., functionally and/or structurally similar to the generator 110, the generator 210, and/or any generator described herein), according to an embodiment. The method 1300 corresponds to different state conditions of the generator. The state conditions can include a state off conditions at 1302, a state engage condition at 1304, a state cut condition at 1306, a state auto off condition at 1308, a certify complete condition at 131, and a state disengage condition at 1312.

At 1302, the state off condition includes the generator in an off mode where energy is not generated by the generator. In some embodiments, the state off condition can include the generator configured to determine whether components of the electrosurgical system are connected to the generator as desired. For example, the generator can determine if an energy delivery element, a ground, and/or the like are connected to the generator. The generator can further determine if the guidewire has a valid electrically EEPROM and/or the ground to have a valid impedance. In some embodiments, if a component is not connected as desired, the generator can generate an alert. For example, in some embodiments, the generator can include a display that when activated can display if components are connected to the generator as desired. When the generator receives a signal indicating that a cut (e.g., tissue puncture) is desired, the method 1300 can continue to 1304. The signal can include a signal from an actuation device such as button, a switch, a footswitch, and/or the like. In some embodiments, the actuation device is on or near the generator, coupled to the generator, and/or on or nearby the electrosurgical device. In some embodiments, multiple actuations devices can be used that are logically coupled together to generate the cut signal. In some embodiments, if the cut signal is activated, a notification (e.g., audio, visual, haptic, etc.) can be generated to indicate the cut signal is activated.

At 1304, the state engage condition is activated after the cut signal is received. The generator can then generate energy for delivery by the electrosurgical device and for monitoring electrical parameters. In some embodiments, the state engage condition is active when an activation switch is constantly activated. If the activation switch, and thus the cut signal, is deactivated, the method 1300 can continue to 1312. After an initial range finding and detection of electrical parameters, the method 1300 continues to 1306 or 1308. In some embodiments, the initial range finding and detection of electrical parameters can occur during a predetermined period of time. In some embodiments, the predetermined period of time is about 25 ms. The method continues to 1306 is an auto off setting is disabled and to 1308 if the auto off setting is enabled.

At 1306, the state cut condition is activated while the electrosurgical device is engaging the tissue. The state cut condition will remain activated until cut signal is no longer activated, after which the method 1300 proceeds to 1312. At 1308, the state auto off condition is activated while the electrosurgical device is engaging the tissue. During the state auto off condition the generator can monitor electrical parameters such as power. In some embodiments, one or more electrical parameter can be used an input to a controller (e.g., proportional-integral-derivative, etc.) to determine if one or more electrical parameter should be increased or decreased. In some embodiments, the generator can monitor the one or more electrical parameter to determine if one or more safety threshold is reached. For example, if the current and/or the impedance exceeds a present safety threshold, the generator can shut off energy by turning off the cut signal so that the method proceeds to 1312. In some embodiments, the one or more safety threshold can be inputted by a user. In some embodiments, the one or more safety threshold can be associated with a derivative (e.g., first derivative, second derivative, etc.) of the one or more parameter and/or an integral of the one or more parameter. In some embodiments, the state auto off condition can continue to 1310 if the magnitude of one or more parameter reaches a target value. In some embodiments, the one or more parameter includes impedance and the target value is a target impedance.

In some embodiments, the state cut condition can include a pre-delay (e.g., prior to the determination of the cutoff, the threshold at which cutoff can occur, the threshold hysteresis, etc.) and/or a post-delay (e.g., how long the generator stays activated after a cut off event is detected). In some embodiments, the pre-delay and the post-delay can be set by the generator using predetermined systems, user inputs, user inputs, and/or the like.

At 1310, the certify complete condition can include determining that if the one or more parameter still reaches the target value after a predetermined period of time. If the one or more parameter does not reach the target value after the predetermined period of time, the method 1300 can return to 1308. After 1310, the method continues to 1312. At 1312, energy form the generator is terminated. In some embodiments, the user can review why the energy was terminated and can reset the generator to return to 1302.

FIGS. 14A-14B depict representative circuits of an electrosurgical device and tissue, according to embodiments. FIG. 14A depicts a series representation of a circuit of a generator delivering energy to an electrosurgical device and tissue. FIG. 14B depicts a parallel model of the circuit. The parallel model models the sheath (e.g., of the electrosurgical device) and the wire and the tissue. The series and the parallel models allow for determining different electrical parameters associated with operating the electrosurgical device which can be used for monitoring a procedure or for altering one or more electrical parameters as desired.

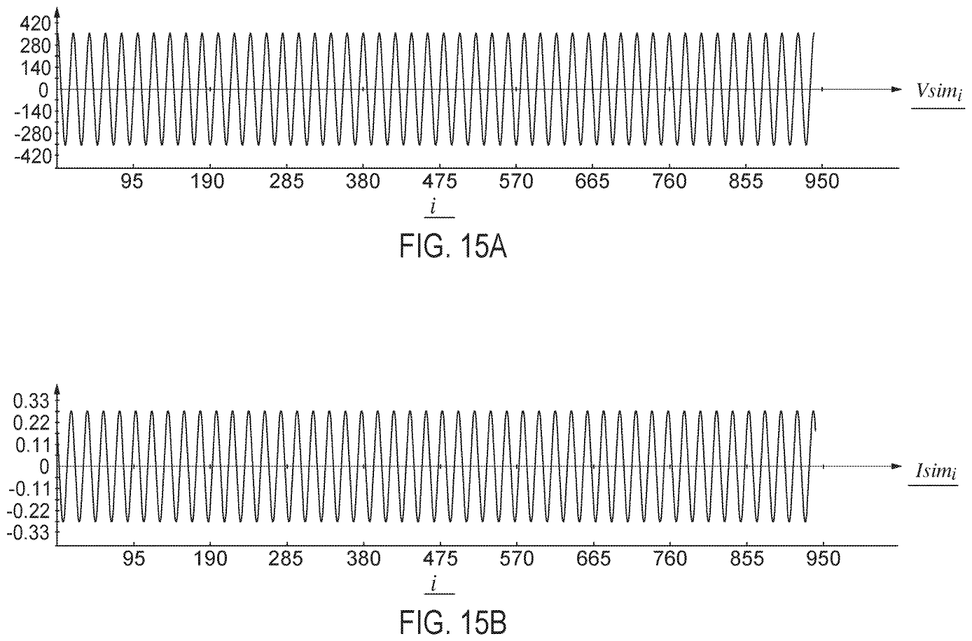

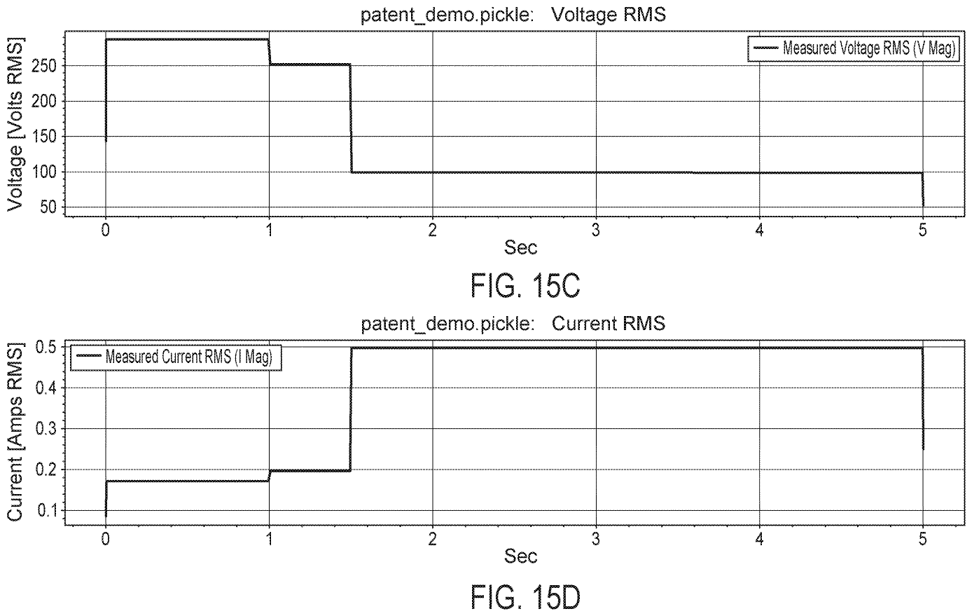

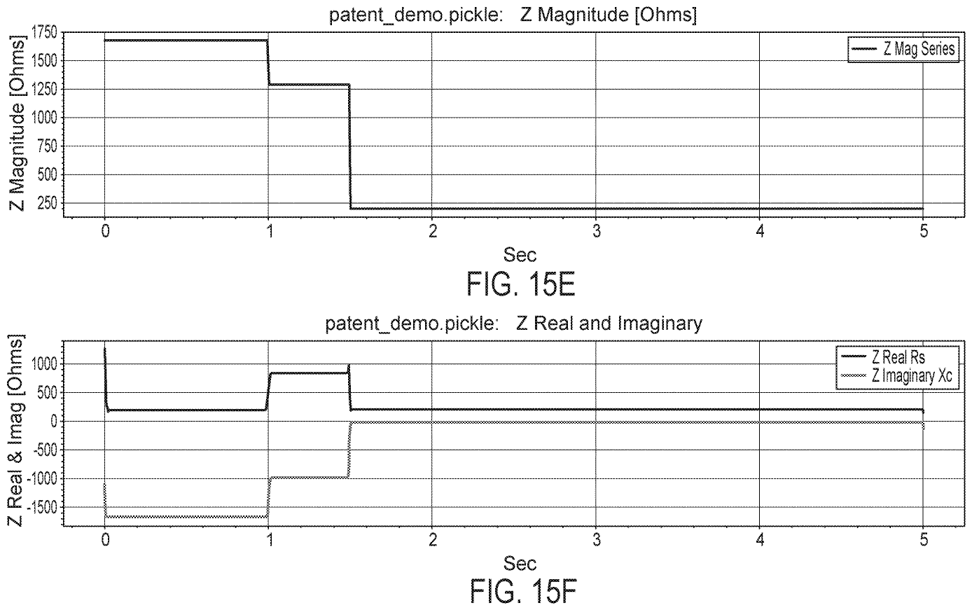

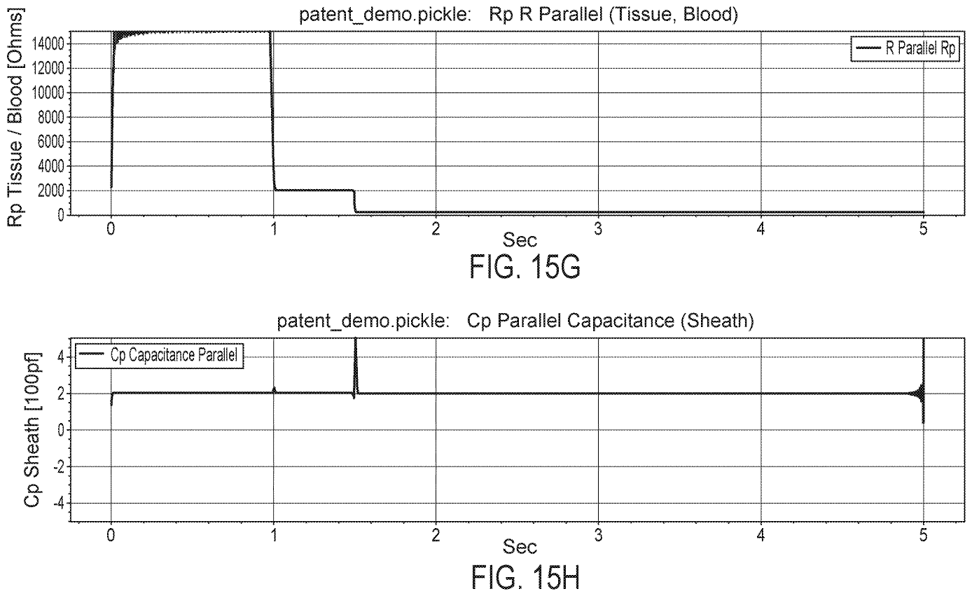

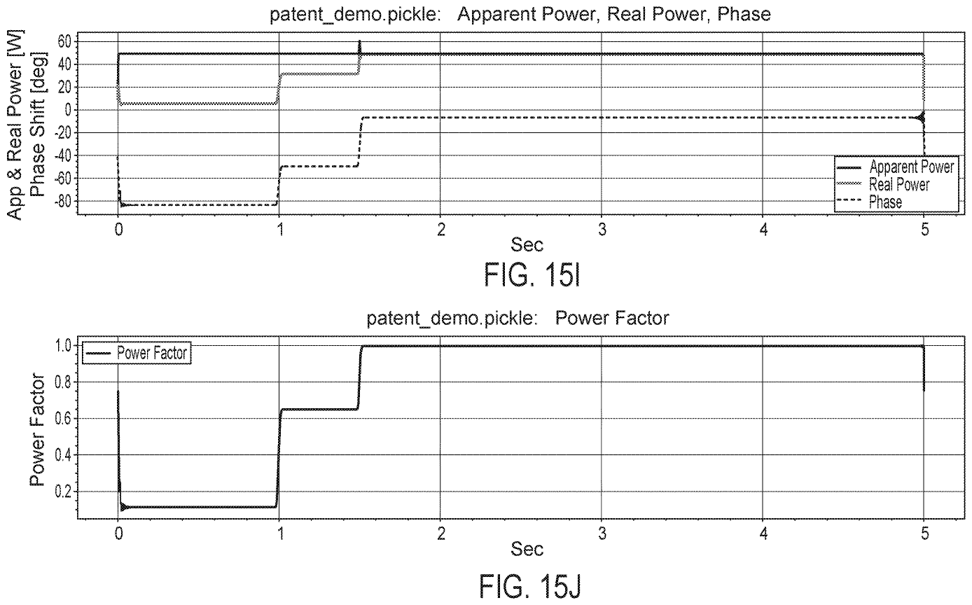

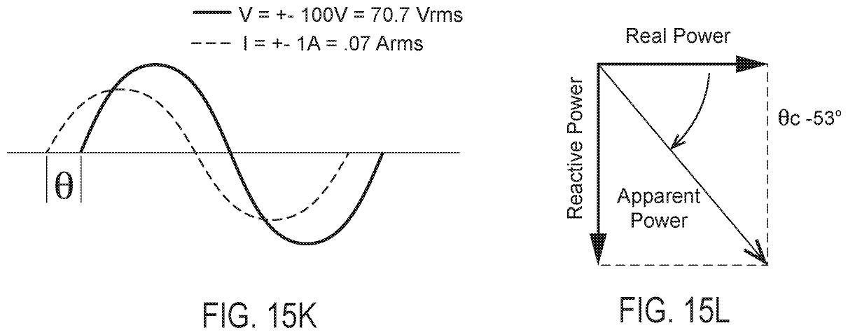

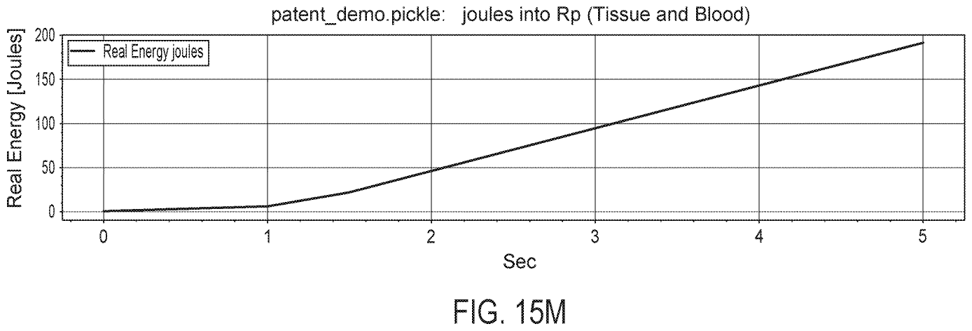

FIGS. 15A-15M depict detected and determined values associated with an electrosurgical device (e.g., structurally and/or functionally similar to any of the electrosurgical devices described herein) engaging tissue, according to embodiments. The FIGS. 15A-15M correspond to the same procedure. During the procedure, a guidewire of the electrosurgical device is in a sheath from t=0 s to about t=1 s (e.g., first state). The guidewire is then in contact with the tissue form about t=1 s to about t=1.5 s (e.g., second state). The guidewire then punctures through the tissue and is in the blood pool of the right atrium from t=1.5 s onward (e.g., third state).

FIGS. 15A-15B depict voltage and current measured from the generator, respectively. The voltage and the current are received as AC signals. FIGS. 15C-15D depict the RMS voltage and the RMS current, respectively, of the voltage and current of FIGS. 15A-15B. As seen in FIG. 15C, the voltage during the first state is at its highest value (e.g., approximately 290 Volts RMS), then decreases to about 250 Volts RMS during the second state, then decreases to about 100 Volts RMS during the third state. Conversely, as seen in FIG. 15D, the current is at its lowest value (e.g., about 0.175 amps RMS) during the first state, increases to about 0.2 amps RMS during the second state, then increases to about 0.5 amps RMS during the third state.

The RMS voltage and current can be used to determine the impedance magnitude, which can be seen in FIG. 15E. The impedance magnitude, during the first state, is about 1700 Ohms, then decreases to about 1300 ohms during the second state, then finally decreases to about 200 during the third state. FIG. 15F depicts the real and imaginary components of the impedance. During the first state, the real impedance is about 200 ohms, and the imaginary impedance is about −1750 ohms. During the second state, the real impedance is about 750 ohms, and the imaginary impedance is about −1000 ohms. During the third state, the real impedance is about 200 ohms, and the imaginary impedance is about zero.

FIG. 15G depicts a graph of the resistance across the guidewire and tissue according to the parallel model of FIG. 14B. During the first state, the resistance is about 15,000 ohms, which decreases to about 2000 ohms during the second state, and further decreases to about 50 ohms during the third state. FIG. 15H depicts the capacitance across the sheath according to the parallel model of FIG. 14B. As the capacitance across the sheath is an inherent property of the sheath, the capacitance remains constant across the first state, second state, and third state.

FIG. 15I depicts a graph of the apparent power, the real power, and the phase. FIG. 15K depicts the relationship between the current, voltage, and the phase angle (θ). The phase angle can be used to determine the real power (e.g., power delivered to the tissue/blood) based on the apparent power. As seen in FIG. 1, the apparent power is a preset value that is constant (e.g., 50 W) during the first state, the second state, and the third state. During the first state, the real power is about 5 W, and the phase is about −85 degrees. During the second state, the real power is about 35 W, and the phase is about −50 degrees. During the third state, the real power is about 50 W, and the phase is about −5 degrees. FIG. 15J depicts the power factor which corresponds to the ratio between the apparent power and the real power. During the first state the power factor is about 0.1. During the second state, the power factor is about 0.65. During the third state the FIG. 15M depicts the energy applied to the tissue and/or the blood. FIG. 15M, measured in cumulative joules of energy delivered during the procedure using the time integral of the real power. During the first state, the energy curve has a first slope that increases to a second slope during the second state and again increases to a third slope in the third state.

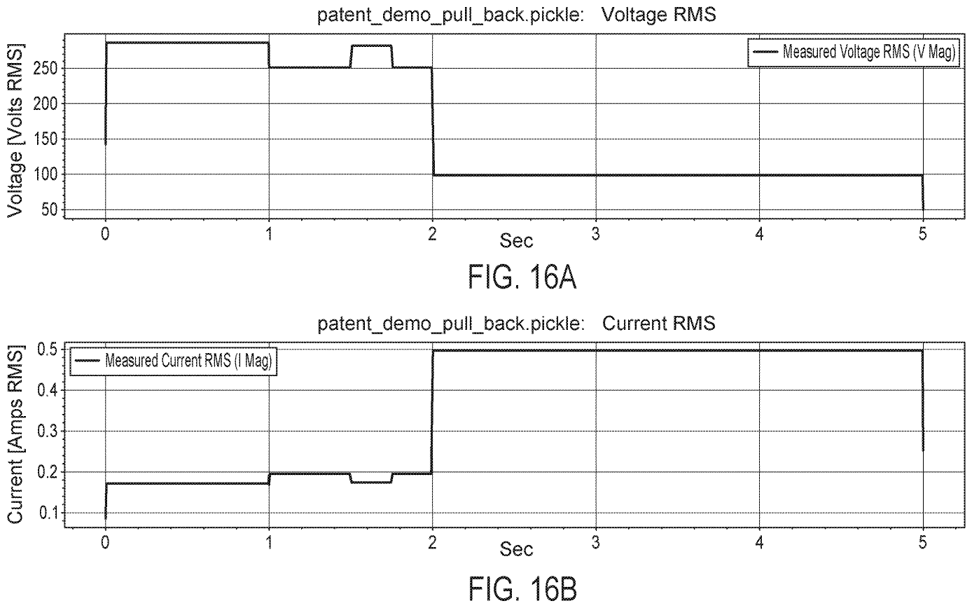

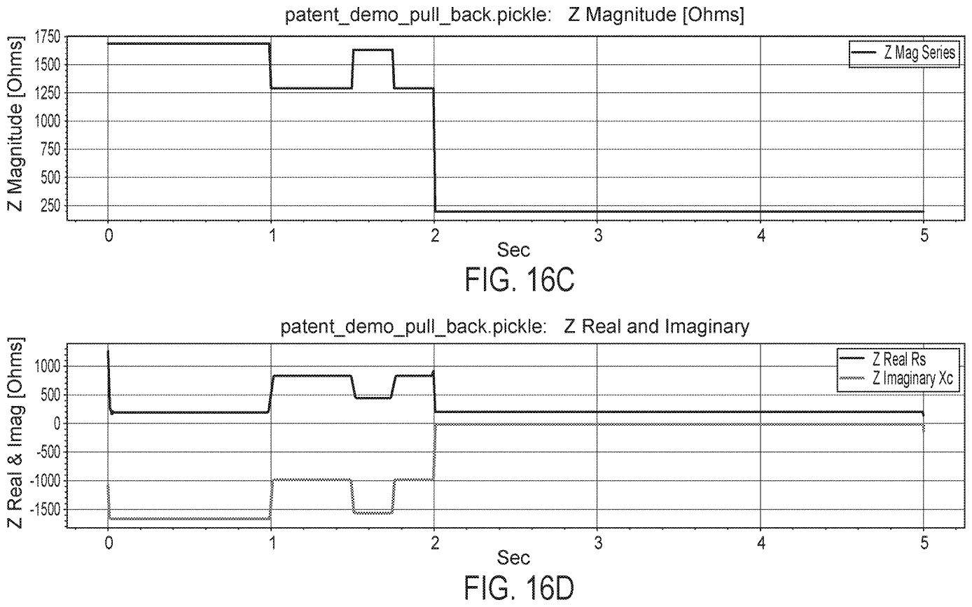

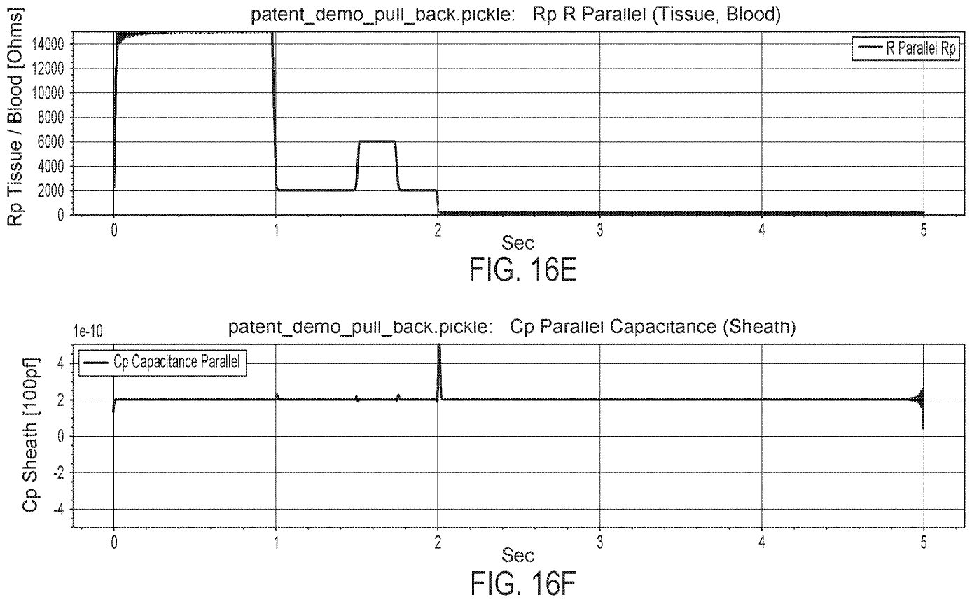

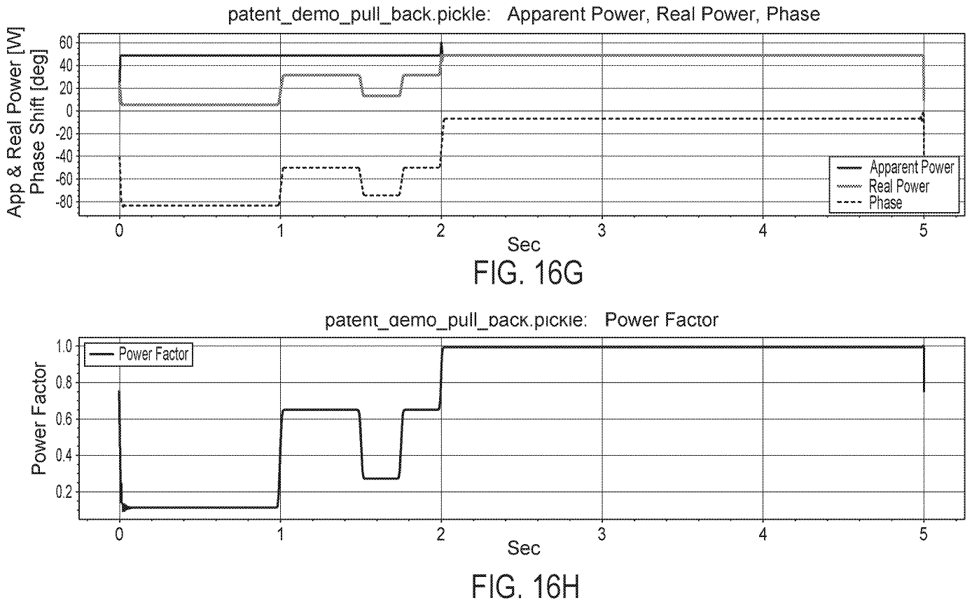

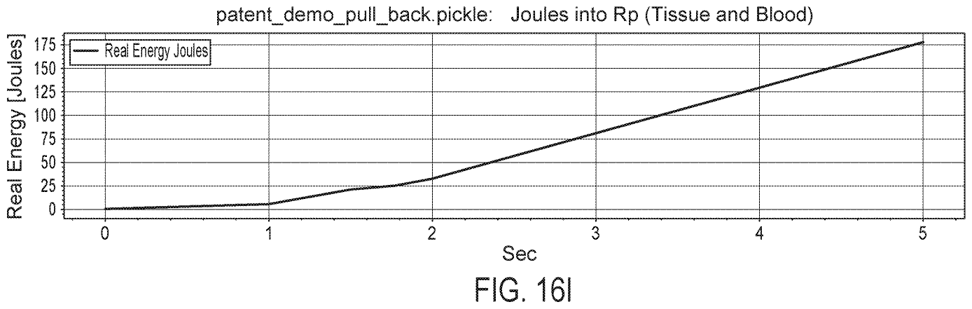

FIGS. 16A-16I depict detected and determined values associated with an electrosurgical device engaging tissue, according to embodiments. The procedure depicted in the graphs of FIGS. 16A-16I are substantially similar to the corresponding graphs in FIGS. 15A-15M, however, the procedure of FIGS. 16A-16I includes a pull back where, during the second state, which lasts from about t=1 s to about t=2 s, the wire is pulled back into the sheath, but not as deep as during the first state (e.g., t=0 s to about t=1 s). The third state of FIGS. 16A-16I is from about t=2 s onwards.

FIGS. 16A-16B depict the RMS voltage and RMS current, respectively. During the pull back during the second state, the voltage RMS increases to about 270 Volts RMS, which is less than during the first state as the guidewire is not as retracted in the sheath. During the pull back, the current RMS decrease to about 0.175 amps RMS.

FIG. 16C depicts the impedance magnitude. During the pull back, the impedance magnitude increases to about 1600 ohms. As seen in FIG. 16D, during the pull back, the real impedance decreases to about 490 ohms and the imaginary impedance decreases to about −1550 ohms. FIG. 16E depicts a graph of the resistance across the guidewire and tissue according to the parallel model of FIG. 14B. During the pull back of the second state, the resistance increases to about 6000 Ohms, which is less than during the first state as the guidewire is not as retracted as in the first state. FIG. 16F depicts a constant capacitance as expected.

FIG. 16G depicts apparent power, real power, and phase. During the pull back, the apparent stays constant, as expected, the real power decreases to about 15 W, and the phase decreases to about −75 degrees. Thus, as seen in FIG. 16H, the power factor decreases to about 0.27 during the pull back. FIG. 16I depicts the total cumulative energy delivered into the blood and/or tissue. During the pull back the slope of the energy curve is smaller than during the other sections of the second state.

It should be understood that the examples and illustrations in this disclosure serve exemplary purposes and departures and variations can be built and deployed according to the teachings herein without departing from the scope of this invention. For example, while systems disclosed herein are shown having a unipolar configuration where the electrical circuit is completed by a remotely located return electrode placed externally on a patient, in alternative embodiments, such systems can have a bipolar configuration where a return electrode is placed internally in a patient, such as, for example, a metallic ring electrode disposed on a sheath, dilator, or catheter.

As used herein, the terms “about” and/or “approximately” when used in conjunction with numerical values and/or ranges generally refer to those numerical values and/or ranges near to a recited numerical value and/or range. In some instances, the terms “about” and “approximately” mean within ±10% of the recited value. For example, in some instances, “about 100 [units]” may mean within ±10% of 100 (e.g., from 90 to 110). The terms “about” and “approximately” may be used interchangeably.

Some embodiments described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Ruby, Visual Basic®, VHDL, Verilog, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

The specific examples and descriptions herein are exemplary in nature and embodiments may be developed by those skilled in the art based on the material taught herein without departing from the scope of the present invention, which is limited only by the attached claims.

1. An apparatus, comprising:
an electrosurgical device including a distal conductive region configured to puncture through a tissue wall; and
a generator configured to couple to the electrosurgical device, the generator including:
a memory; and
a processor operatively coupled to the memory and being configured to:
deliver a voltage waveform to the distal conductive region of the electrosurgical device;
while delivering the voltage waveform, monitor one or more electrical parameters via the distal conductive region;
determine a plurality of states of the distal conductive region over a period of time based on the one or more electrical parameters;
determine whether at least one state of the plurality of states satisfies a condition indicative of the one or more electrical parameters being outside of a predefined range; and
in response to determining that the at least one state of the plurality of states satisfies the condition, terminate the delivery of the voltage waveform to the distal conductive region.

2. The apparatus of claim 1, wherein the condition is associated with puncturing through the tissue wall.

3. The apparatus of claim 1, wherein the electrosurgical device further includes an electrosurgical interface, the electrosurgical interface including an actuator configured to be actuated to generate a signal to initiate the delivery of the voltage waveform,
the processor further configured to receive, when the actuator is actuated, the signal from the electrosurgical interface,
the processor configured to initiate the delivery of the voltage waveform in response to receiving the signal.

4. The apparatus of claim 1, wherein the processor is further configured to measure a voltage and a current flowing to the distal conductive region over the period of time,
the processor configured to monitor the one or more electrical parameters including an impedance based on the voltage and the current.

5. The apparatus of claim 4, wherein the processor is configured to determine the plurality of states of the distal conductive region based on the impedance over the period of time.

6. The apparatus of claim 1, wherein the processor is configured to determine whether the at least one state of the plurality of states satisfies the condition indicative of puncturing through the tissue wall by determining whether at least a subset of states of the plurality of states follows a predefined state transition indicative of puncturing through the tissue wall.

7. The apparatus of claim 1, wherein the processor is configured to determine at least one state of the plurality of states of the distal conductive region based on the one or more electrical parameters and the state of the plurality of state immediately preceding the at least one state.

8. The apparatus of claim 1, wherein the distal conductive region has a length of at least about 3 mm.

9. An apparatus, comprising:
an electrosurgical device including a distal conductive region configured to puncture through a tissue wall, the distal conductive region having a length of at least about 1 cm; and
a generator configured to couple to the electrosurgical device, the generator including:
a memory; and
a processor operatively coupled to the memory and being configured to:
deliver a voltage waveform to the distal conductive region of the electrosurgical device;
while delivering the voltage waveform, monitor an electrical parameter of an circuit including the distal conductive region;
determine, based on the electrical parameter, whether a condition indicative of the electrical parameter being outside of a predefined range; and
in response to determining that the condition is satisfied, terminate the delivery of the voltage waveform to the distal conductive region.

10. The apparatus of claim 9, wherein the condition is associated with puncturing through the tissue wall.

11. The apparatus of claim 9, wherein the electrical parameter is impedance.

12. The apparatus of claim 11, wherein the processor is configured to determine whether the condition is satisfied by determining whether a magnitude of the impedance is less than a predetermined threshold for a predetermined period of time.

13. The apparatus of claim 11, wherein the processor is further configured to determine, based on the impedance, which one of a plurality of states is a current state of the distal conductive region.

14. The apparatus of claim 13, wherein the plurality of states includes:
a state of being disposed within an insulative sheath, a state of engaging the tissue wall, and a state of being disposed in a blood pool.

15. A system, comprising:
an electrosurgical device including a distal conductive region configured to deliver energy to cross through a tissue wall, the distal conductive region having a length of at least about 1 cm; and
a generator configured to couple to the electrosurgical device, the generator including:
a memory; and
a processor operatively coupled to the memory and being configured to:
deliver a voltage waveform to the distal conductive region to cause the delivery of energy;
while delivering the voltage waveform, measure voltage and current signals over time via the distal conductive region;
determine, based on the voltage and current signals, an impedance over time associated with the distal conductive region;
determine, based on the impedance over time and during a delivery of energy to the distal conductive region, a state of the distal conductive region, the state corresponding to a position of the distal conductive region relative to a surface of the tissue wall; and
terminate, when the state of the distal conductive region is indicative of a failure mode or a successful crossing of the tissue wall, the delivery of the voltage waveform to the distal conductive region.

16. The system of claim 15, wherein the state is one of: a state of the distal conductive region being disposed within an insulative sheath, a state of the distal conductive region engaging the tissue wall, a state of the distal conductive region being disposed in a blood pool, a state of the distal conductive region deflecting from the tissue wall, and a state of the distal conductive region contacting a portion of tissue that is not the tissue wall.

17. The system of claim 15, wherein the processor is further configured to determine, based on the voltage and current signals, a phase associated with the energy delivered by the distal conductive region.

18. The system of claim 17, wherein the processor is configured to determine the phase by:
determining real and complex voltage from the voltage signal and real and complex current from the current signal; and
determining the phase, based on the complex voltage and the complex current.

19. The system of claim 15, wherein the processor is further configured to determine at least one of: apparent power, real power, or a power factor.

20. The system of claim 15, wherein the processor is configured to determine that the state of the distal conductive region is indicative of the successful crossing of the tissue wall by determining that a magnitude of the impedance remains less than a predetermined threshold for a predetermined period of time.

21.-35. (canceled)
